(19) United States
(12) Patent Application Publication
(10) Pub. No.: US 2015/0004113 A1
(43) Pub. Date: Jan. 1, 2015

(54) SILOXANE POLYMERS WITH A CENTRAL POLYSILOXANE POLYMER BLOCK WITH TERMINAL ORGANOFUNCTIONAL RADICALS COMPRISING UREA AND/OR CARBAMATE GROUPS AND AMINO ACID RADICALS

(71) Applicant: EVONIK INDUSTRIES AG, Essen (DE)

(72) Inventors: Helmut Ritter, Wuppertal (DE); Berit Knudsen, Erkrath (DE); Wilfried Knott, Essen (DE); Frauke Henning, Essen (DE); Christian Hartung, Essen (DE); Thiemo Mennenga, Haan (DE)

(21) Appl. No.: 14/313,288

(22) Filed: Jun. 24, 2014

(30) Foreign Application Priority Data
Jul. 1, 2013 (DE) 102013106906.1

(51) Int. Cl. C08G 77/388; A61Q 5/12; A61K 8/898

(52) U.S. Cl. CPC C08G 77/388; A61K 8/898; A61Q 5/12
USPC 424/70.12; 525/474; 560/355

(57) ABSTRACT

The invention relates to siloxane polymer comprising a central polysiloxane polymer block B with organofunctional radicals, which are terminal or bonded laterally on the polymer block, comprising IPDI and amino acid derivatives which are covalently bonded via a hydrophobic or hydrophilic linker group Q1′, Q2′, and to compositions comprising these siloxanes. Furthermore, processes for their preparation and their use are disclosed.

The present application claims priority from German Patent Application No. DE 10 2013 106 906.1 filed on Jul. 1, 2013, the disclosure of which is incorporated herein by reference in its entirety.

### FIELD OF THE INVENTION

The invention relates to siloxane polymers comprising a central polysiloxane polymer block B with side-comb-positioned organofunctional radicals, which are terminal or bonded laterally on the polymer block, comprising IPDI and amino acid derivatives, which are covalently bonded via a hydrophobic or hydrophilic linker group Q1′, Q2′, and to compositions comprising these siloxanes. Furthermore, processes for their preparation and their use are disclosed.

For the treatment and modification of the properties of textile fibres, and also keratin fibres as well as for the skin, the profile of properties and the achieved effect in the area of hair care, body care or treatment of textiles can be considerably improved by adding modified siloxanes as additives.

For example, essential product properties can be improved considerably by adding modified silicones. Mention is to be made of the improved suppleness of creams, the skin feel, the shine of hair or its combability, and also the water resistance of sun creams. Thus, aminofunctional siloxanes can have a shine- and feel-conveying effect in textiles or hair care. Controlling these properties is possible by varying lateral aminoalkyl radicals of the siloxanes by varying the nitrogen content of the aminoalkyl radical or adjusting the molecular weight of the siloxane.

In order to expand the application spectrum of the nitrogen-containing siloxanes, there is a need for further nitrogen-containing siloxanes. A particular focus here is on siloxanes whose backbone deliberately has regions with different properties. Thus, there is a need for siloxanes which have hydrophobic regions and at the same time regions which are hydrophilic or water-soluble. There is a particular need for siloxanes which are able to add via hydrogen bridge bonds onto natural surfaces, such as keratin fibres or else textile natural fibres. Particularly preferably, the siloxanes should be able to be adjusted as regards their hydrophilicity and/or hydrophobicity. Preferably, the aim is to develop a siloxane or a mixture of siloxanes which can be used either as additive in cosmetic formulations or in the treatment of textiles in order to preferably improve the combability of the fibres and at the same time permits improved adhesion to the fibres by being able to adhere to the surface of keratin, synthetic or natural textile fibres with the two terminal regions or at least to a plurality of regions via hydrogen bridge bonds. One object consisted in providing a Gemini surfactant (bis-surfactant or double surfactant) which is suitable for use in the cosmetics sector, in the textile industry, in detergent formulations, and also as additive for influencing the surface properties of coatings, impression materials etc., and can for example have a positive influence on the spreading of water drops etc.

It is noted that citation or identification of any document in this application is not an admission that such document is available as prior art to the present invention.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

It is further noted that the invention does not intend to encompass within the scope of the invention any previously disclosed product, process of making the product or method of using the product, which meets the written description and enablement requirements of the USPTO (35 U.S.C. 112, first paragraph) or the EPO (Article 83 of the EPC), such that applicant(s) reserve the right to disclaim, and hereby disclose a disclaimer of, any previously described product, method of making the product, or process of using the product.

### OBJECTS OF THE INVENTION

The objects are achieved by the siloxane according to the invention and compositions comprising these corresponding to the features of Claims 1 and 22 and 23 relating to the composition according to the invention comprising at least one siloxane according to the invention, and also by the process for the preparation according to Claim 15 and also the formulation according to the invention corresponding to the features of Patent Claim 24.

Surprisingly, the objects were achieved by providing siloxane polymers which have a central polysiloxane polymer block B and at least two terminal or at least one terminal and at least one lateral, organofunctional group, which are derived in each case from a reaction of an isocyanate group of diisocyanates with amino acids, amino acid derivatives or salts thereof, where the formed amino acid isocyanate is bonded to the polysiloxane via a linker (Q1′, Q1′AH, Q2′, Q2′AH). Suitable linkers are preferably alkylene, —(CH₂)ₙ— (n=2 to 200), aryl, arylalkylene, optionally with heteroatoms O, N and/or S, compounds containing aminoalkylene, quarternary aminoalkylene, allyl, ester, amide, anhydride, urea, (meth)acrylate groups, and substituted and unsubstituted polyethers, in particular polyethylene glycol [EO]v, propylene glycol—[PO]w or [EO]v[PO]w.

### SUMMARY OF THE INVENTION

The invention provides at least one siloxane polymer of the general Formula I comprising a central polysiloxane polymer block B,

(i) which is substituted with organofunctional radicals, the organofunctional radicals preferably comprise an alkylene radical with 1 to 22 carbon atoms and/or a phenyl group or a polyether,
(ii) the polymer block B has linear and/or branched structures with at least two difunctional siloxane units,
(iii) the polymer block B has on at least two terminals silicon atoms or at least one terminal and at least one lateral silicon atom on the siloxane units of polymer block B, the organofunctional radicals -Q1 and -Q2, where the radicals are identical or different,

Q2-B-Q1 (I)

where -Q1 corresponds to the general formula IIa and -Q2 corresponds to the formula IIb, which are independently identical or different,

-Q1=-Q1′-A-(C═O)-D-Q1″-A′-(C═O)-D′-Q1* (IIa)

-Q2=-Q2′-A-(C═O)-D-Q2″-A′-(C═O)-D′-Q2* (IIb)
where A is —NH—, —O— or —S— and D is —NH— in each case independently in formulae IIa and IIb,
where A′ is —NH— and D′ is —NH—, —O— or —S— in each case independently in IIa and IIb, where each radical Q1 and Q2 of the formulae IIa or IIb has at least one bivalent urea group and one further bivalent urea or carbamate, group
with Q1′ and Q2′ in each case independently comprising a bivalent hydrocarbon radical with 6 to 200 carbon atoms optionally comprising at least one heteroatom O, N or S, a bivalent radical comprising aryl, arylalkyl groups optionally comprising at least one heteroatom O, N or S or polyether radicals containing alkyl, aryl or alkyl and aryl groups,
with Q1″ and Q2″ in each case independently comprising a bivalent linear, branched and/or cyclic alkyl radical having 4 to 200 carbon atoms, in particular cyclohexenyl radical based, like from the reaction of IPDI or a bivalent radical comprising an aryl and/or arylalkyl radical with 6 to 200 carbon atoms,
with -D′-Q1* and -D′-Q2* with D′ as defined above, and where -D′-Q1* and -D′-Q2* in each case independently comprise radicals which are derived from an amino acid, an amino acid derivative or salts thereof or mixtures thereof.

Preferably, B corresponds to the formulae IIIa or IIIb. Preferably, Q1′ or Q2′ is in each case independently a bifunctional linear, branched or cyclic alkylene radical with 1 to 22 carbon atoms, a bifunctional aryl, arylalkylene radical with 6 to 30 carbon atoms or polyether. And preferably with Q1″ and Q2″, which are in each case independently bifunctional linear, branched or cyclic alkylene group with 1 to 22 carbon atoms or bifunctional aryl, arylalkylene radicals with 6 to 30 carbon atoms.

### DETAILED DESCRIPTION OF EMBODIMENTS

It is to be understood that the figures and descriptions of the present invention have been simplified to illustrate elements that are relevant for a clear understanding of the present invention, while eliminating, for purposes of clarity, many other elements which are conventional in this art. Those of ordinary skill in the art will recognize that other elements are desirable for implementing the present invention. However, because such elements are well known in the art, and because they do not facilitate a better understanding of the present invention, a discussion of such elements is not provided herein.

According to a particularly preferred embodiment, the siloxane polymers according to the invention of the general formulae I, IIa and IIb have as -D′-Q1* and -D′-Q2* in each case independently radicals which are derived from an amino acid, an amino acid derivative or salts thereof, and D′ where D′ is —NH— in each case independently in -D′-Q1* and -D′-Q2* is in the alpha position relative to an ester, carboxy or carbonyl group of the amino acid derivative, where the ester comprises alkyl esters with 1 to 25 carbon atoms or aryl esters. Particularly preferred esters are the methyl or ethyl esters of the alpha-amino acids, derivatives of the alpha-amino acids, and the protonated forms of the alpha-amino acids, of the esters or derivatives. The basic alpha-amino acids whose esters and/or amides are used for preparing the siloxanes or in Q1, Q2 are in accordance with the invention. Suitable basic alpha-amino acids are the amino acids with basic, protonatable side chains, such as lysine, histidine and arginine. Particular preference is given to histidine and arginine. Preference is likewise also given to the polar/neutral HS—, HO—, H₂N-side-group-substituted amino acids and the acidic amino acids glutamic acid and aspartic acid.

The invention likewise provides siloxane polymers of the formula I with the radicals -Q1 and -Q2, where the radicals -D′-Q1* and -D′-Q2* are in each case independently derived from amino acids, amino acid derivatives or their salts, in particular the hydrochlorides or other physiologically compatible salts, comprising
nonpolar amino acids comprising alanine, valine, methionine, leucine, isoleucine, proline, tryptophan, phenylalanine,
basic amino acids comprising lysine, arginine, histidine,
polar and neutral amino acids tyrosine, threonine, glutamine, glycine, serine, cysteine, asparagine and/or
acidic amino acids selected from glutamic acid and aspartic acid,
and their mono-, dicarboxylic acid esters, amides with primary amino groups of the amino acids, amides of the carboxylic acid groups of the amino acids and/or esters with the primary hydroxyl groups, thioesters of the HS group or amides with secondary amino groups of the amino acids. The invention also provides siloxane polymers without a lysine derivative, in particular without lysine derivatives based on amides with fatty acids. Suitable carbamate groups are either —O—(C═O)—NH— or —S—(C═O)—NH— groups, which can also be referred to as thiocarbamates (thiolurethane).

Siloxanes with the following substitution pattern have particularly advantageous properties as regards an improved combability of keratin fibres, in particular hair, shown according to the test disclosed below. Consequently, the invention further provides at least one siloxane polymer or a composition comprising at least a corresponding siloxane polymer or a mixture of these, comprising the radicals -Q1 and -Q2 of the formula I where A is —O—, D is —NH—, A′ is —NH— and D′ is —NH—, —O— or —S—, in particular where D′ is —NH, where the radicals -D′-Q1* and -D′-Q2* are in each case independently derived from amino acids, amino acid derivatives, salts thereof, protonated amino acids or derivatives thereof, in particular comprising nonpolar amino acids comprising alanine, valine, methionine, leucine, isoleucine, proline, tryptophan, phenylalanine, basic amino acids comprising lysine, arginine, histidine, polar and neutral amino acids comprising tyrosine, threonine, glutamine, glycine, serine, cysteine, asparagine and/or the acidic amino acids selected from glutamic acid and aspartic acid, and their mono-, dicarboxylic acid esters, amides with primary amino groups of the amino acids, amides of the carboxylic acid groups of the amino acids and/or esters with the primary hydroxyl groups or thioesters of the HS group, and salts thereof. In all general formulae, it is the case that the following symbol [symbol] indicates a bonding site/monovalent bonding site to which—not shown—an atom, a group or radical is covalently bonded. In the case of the substituted cyclohexyl radicals based on the diisocyanate isophorone radicals, the NH(C═O)A- or NH(C═O)D′- radicals are bonded onto the bonding sites [symbol] In the formulae IIIa, IIIb, the radicals Q2- and -Q1 are covalently bonded to

[symbol]

According to a particularly preferred embodiment, the invention provides at least one siloxane polymer of the general formula I or mixtures of these in which the polymer block B corresponds at least to one of the general formulae IIIa or IIIb, where B is

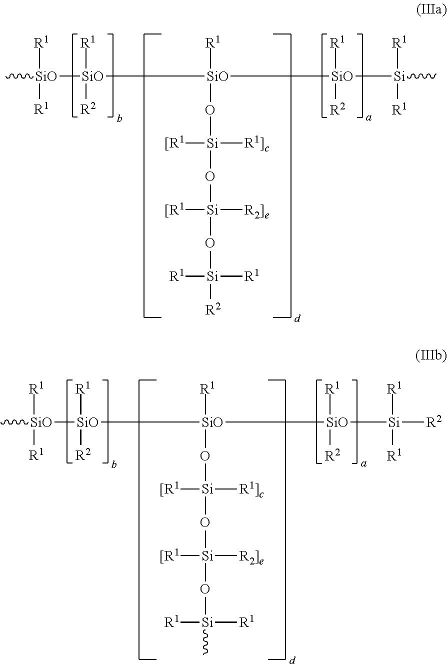

where a, b, c, d and e in formulae IIIa and IIIb are in each case independently an integer
where a is from 1 to 200, in particular 2 to 150, preferably 2 to 100, particularly preferably 5 to 100, preferably 20 to 100,
where b is from 0 to 200, in particular 2 to 150, preferably 2 to 100, particularly preferably 5 to 100, preferably 20 to 100,
where c is from 0 to 200, in particular 2 to 150, preferably 2 to 100, particularly preferably 5 to 50, preferably 5 to 20,
where d is from 0 to 200, in particular 2 to 150, preferably 2 to 100, particularly preferably 5 to 100, preferably 20 to 100,
where e is from 0 to 200, in particular 2 to 150, preferably 2 to 100, particularly preferably 5 to 50, preferably 5 to 20,
where (a+b+c+d+e ) is greater than or equal to 1, preferably greater than or equal to 20, and where R′ in formula IIIa or IIIb is in each case independently identical or different, where R¹ comprises alkyl radicals with 1 to 22 carbon atoms, preferably 1 to 4 carbon atoms, particularly preferably 1, 2, 3, 4, 6 carbon atoms or phenyl radicals,
where R² in formula IIIa or IIIb is alkyl radical with 1 to 22 carbon atoms, preferably 1 to 4 carbon atoms, particularly preferably 1, 2, 3, 4, 6 carbon atoms, an alkyl radical with at least one heteroatom selected from N, O, S, such as an alkylamine, alkylcarboxylic acid, alkylcarboxamide, alkylcarboxylic anhydride, (meth)acrylate, phenyl radical or a radical of the formula
-Q1′-A-(C═O)-D-Q1″-NH₂ and/or Q2′-A-(C═O)-D-Q2″-NH₂. Particularly preferably, R¹ and R² are selected from alkyl groups with 1, 2, 3 or 4 carbon atoms, in particular from methyl groups or at least one R² is an aminoalkyl group or a quaternary alkylamine.

In particularly preferred siloxane polymers of formula I, IIIa and/or IIIb, the indices b, c, d and e are 0 and a is 2 to 200, preferably 20 to 100, in particular a is 30 or 80 with a variation of plus/minus 5.

According to the invention, the radicals -Q1 and -Q2 in the general formula I are independently selected from

-Q1=-Q1′-A-(C═O)-D-Q1″-A′-(C═O)-D′-Q1* (IIa)

-Q2=-Q2′-A-(C═O)-D-Q2″-A′-(C═O)-D′-Q2* (IIb)

a) where A is —O—, D is —NH—, A′ is —NH— and D′ is —NH—,
b) where A is —NH—, D is —NH—, A′ is —NH— and D′ is —NH—,
c) where A is —S—, D is —NH—, where A′ is —NH— and D′ is —NH—,
d) where A is —O—, D is —NH—, A′ is —NH— and D′ is —O—,
e) where A is —NH—, D is —NH—, A′ is —NH— and D′ is —O— or
f) where A is —S—, D is —NH—, where A′ is —NH— and D′ is —O—,
g) where A is —O—, D is —NH—, A′ is —NH— and D′ is —S—,
h) where A is —NH—, D is —NH—, A′ is —NH— and D′ is —S— or
i) where A is —S—, D is —NH—, where A′ is —NH— and D′ is —S—,
where a) where A is —O—, D is —NH—, A′ is —NH— and D′ is —NH— is particularly preferred.

Particularly preferred diisocyanates and urethanes derived therefrom have proven to be in the radicals -Q1 and -Q2 of the formula I the bivalent radicals -Q1″- and -Q2″- which are independently selected from bivalent, linear, branched or cyclic alkylene radicals with 4 to 25 carbon atoms, in particular with 6, 7, 8, 9, 10, 11, 12, 13 or 14 carbon atoms, preferably hexylene (—CH₂)₆, heptylene, bivalent 2,4-toluolyl, diphenylmethane, polymeric diphenylmethane, 3,5,5-trimethyl-1-methylene-3-ethylene-cyclohexane derived from the reaction of IPDI or 4,4′-dicyclohexylene. According to the invention, particular preference is given to using isophorone diisocyanates (IPID) which, on account of the structural isomerism, permit good control of the process since an isocyanate group is more reactive and thus the formation of high molecular weight polymers can be avoided. Consequently, the process is very readily reproducible and the siloxanes are obtainable with defined molecular weight.

Siloxane polymers according to the invention can preferably comprise in the radicals -Q1 and -Q2 of the formula (I), at least as one of the bivalent radicals -Q1″- and -Q2″-, independently a bivalent cyclohexane-containing radical selected from the formulae Va and Vb

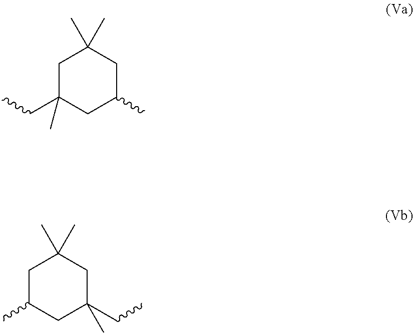

in particular Q2″- is a bivalent cyclohexane-containing radical of the formula Va and -Q1″ is of the formula Vb. Here, on account of the different reactivity of the isocyanate groups, the siloxane polymer, in particular of the formula I, preferably with the structure shown, in particular of the formulae Ia, Ib, Ia*, Ib*, is formed as main product by the process according to the invention according to variant a), process variant b) leads preferentially to the formation of the siloxane polymers of the formula Ic.

The linker (-Q1′-A-, -Q2′-A-) is preferably derived from an olefinic alcohol with 3 to 200 carbon atoms, preferably with 3 to 25 carbon atoms, optionally with at least one heteroatom comprising N, O or S. Likewise preferred in the radicals -Q1 and -Q2 of the formula I, the bivalent radicals -Q1′- and -Q2′-, are selected from alkylene radicals with 3 to 22 carbon atoms optionally with at least one heteroatom comprising N, O or S, in particular —(CH₂)ₙ— where n is from 3 to 22 optionally with at least one heteroatom comprising N, O or S, preference also being given to hexylene (—CH₂)—, heptylene (—CH₂)₇—, octylene (—CH₂)₈—, nonylene (—CH₂)₉—, decylene (—CH₂)₁₀—, undecylene (—CH₂)₁₁—, dodecylene (—CH₂)₁₂—, or with at least one heteroatom, such as alkylene-CO—, such as alkylene-CO—, based on the reaction of 10-undecenoic acid, 3-butenoic acid, acrylic acid, methacrylic acid and 5-hexenoic acid, alkylene-O(CO)-alkylene, alkylene-(CO)O-alkylene, alkylene-NH(CO)-alkylene, alkylene-(CO)NH-alkylene, alkylene-NH(CO)NH-alkylene, alkylene-NH(CO)O-alkylene, or from polyether radicals containing alkyl, aryl or alkyl and aryl groups and of the formulae IVa or IVb where Q1′ and Q2′ are in each case independently

-T-O—(CH₂—CH₂—O—)ₓ—(CH₂—CH(R#)O—)_{y}—(SO)—R″ (IVa)

-T-O—(CH₂—CH₂—O—)ₓ—(CH₂—CH(R#)O—)_{y}—R″ (IVb),

where T=bivalent hydrocarbon radical with 2 to 4 carbon atoms, where x=0 to 200, in particular with 0 to 100, preferably with 0 to 50, y=0 to 200, in particular with 0 to 100, preferably with 0 to 50, where x and y are integers with the proviso that x or y is at least 1, where R# is hydrogen or methyl, R″ is hydrogen or alkylene, (CH₂)₂—, (CH(CH₃))CH₂—, methylene, polymethylene or —(CH₂)₃—, in particular —CH₂—CH₂—, and preferably where T=-(CH₂)₂— or —(CH₂)₃—. Where R″ is hydrogen in a starting material or intermediate and linear or branched alkylene in an intermediate or end product.

Particularly preferred bivalent radicals -Q1″- and -Q2″- are isophorone derivatives, cyclohexylene-containing radicals and polymethylene, such as hexamethylene.

Subsequent siloxane polymers are particularly preferred siloxane polymers and are selected from siloxane polymers of the formula Ia and Ib

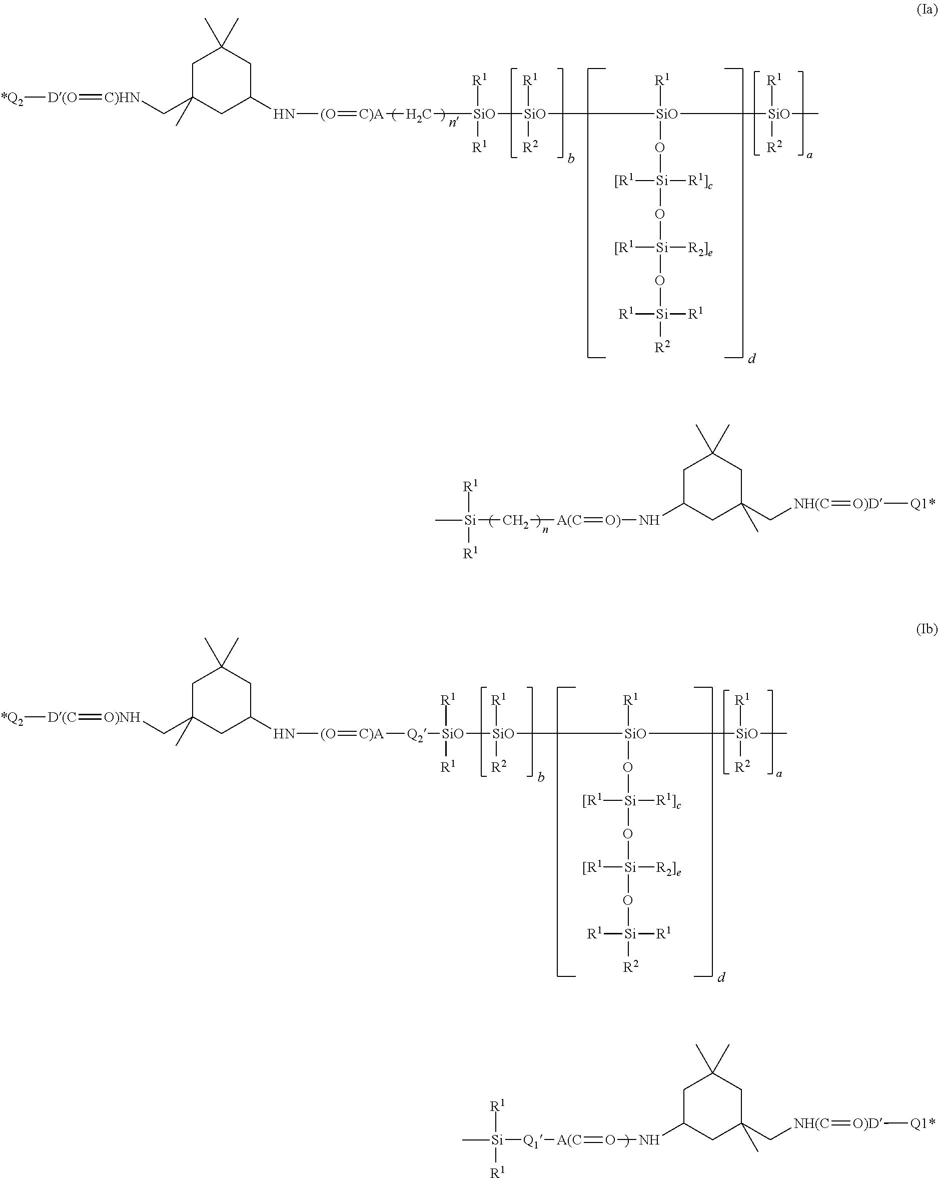

where n or n′ are in each case independently selected from an integer from 2 to 40, in particular with 3 to 22, where a is from 1 to 200, where b is from 0 to 200, where c is from 0 to 200, where d is from 0 to 200, where e is from 0 to 200 and where R′ in formula Ia and Ib is in each case independently identical or different, where R¹ comprises alkyl radicals with 1 to 4 carbon atoms or phenyl radicals, where R² is an alkyl radical with 1 to 22 carbon atoms, preferably 1 to 4 carbon atoms, in particular 1, 2, 3, 4, 6 carbon atoms, an alkyl radical with at least one heteroatom selected from N, O, S, preferably alkylamine, glycidyloxyalkyl radical or phenyl radical and where -D′-Q1* and -D′-Q2* are in each case independently derived from amino acids, amino acid derivatives or salts thereof, and in formula Ib where Q1′ and Q2′ are in each case independently a linear, cyclic, branched alkylene with 2 to 40 carbon atoms or a formula IVa or IVb

-T-O—(CH₂—CH₂—O—)ₓ—(CH₂—CH(R#)O—)_{y}—(SO)—R″ (IVa)

-T-O—(CH₂—CH₂—O—)ₓ—(CH₂—CH(R#)O—)_{y}—R″ (IVb)

where T=bivalent hydrocarbon radical with 2 to 4 carbon atoms, where x=0 to 200, in particular with 0 to 150, 1 to 150, 5 to 150, 5 to 100, y=0 to 200, in particular with 0 to 150, 1 to 150, 5 to 150, 5 to 100, where x and y are integers with the proviso that x or y is at least 1, where R# is hydrogen or methyl, R″ is alkylene, —(CH₂)₂—, —(CH(CH₃))CH₂—, methylene, polymethylene or —(CH₂)₃—, in particular —CH₂—CH₂—, ethylene, in particular where T=-(CH₂)₂— or —(CH₂)₃—,
R² can in part have the meaning of the radicals R¹ and the other radicals R² can, independently of one another, be radicals of the formulae Id, R²=-M-Z* A″ (Ic), where radicals R² are in each case a radical of the formula -M-Z⁺ A⁻, Z⁺ is a radical of the formula Id

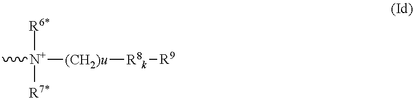

R⁶*, R⁷* are in each case identical or different alkyl radicals with 1 to 22 carbon atoms or alkenyl radicals with 2 to 22 carbon atoms, in which the alkyl or alkenyl radicals can have hydroxyl groups,
where R⁸ is —O—(C═O)— or —NH(C═O)—,
R⁹ can be a monovalent hydrocarbon radical with 1 to 22 carbon atoms, or
u=0 to 6 in formula Id,
k=0 or 1 in formula Id
M is a divalent hydrocarbon radical with at least 4 carbon atoms which can have a hydroxyl group and which can be interrupted by one or more oxygen atoms,
A⁻ is an inorganic or organic anion which stems from a customary physiologically compatible acid HA.

One embodiment of the invention comprises siloxane polymers selected from siloxane polymers of the formula Ia* and Ib*

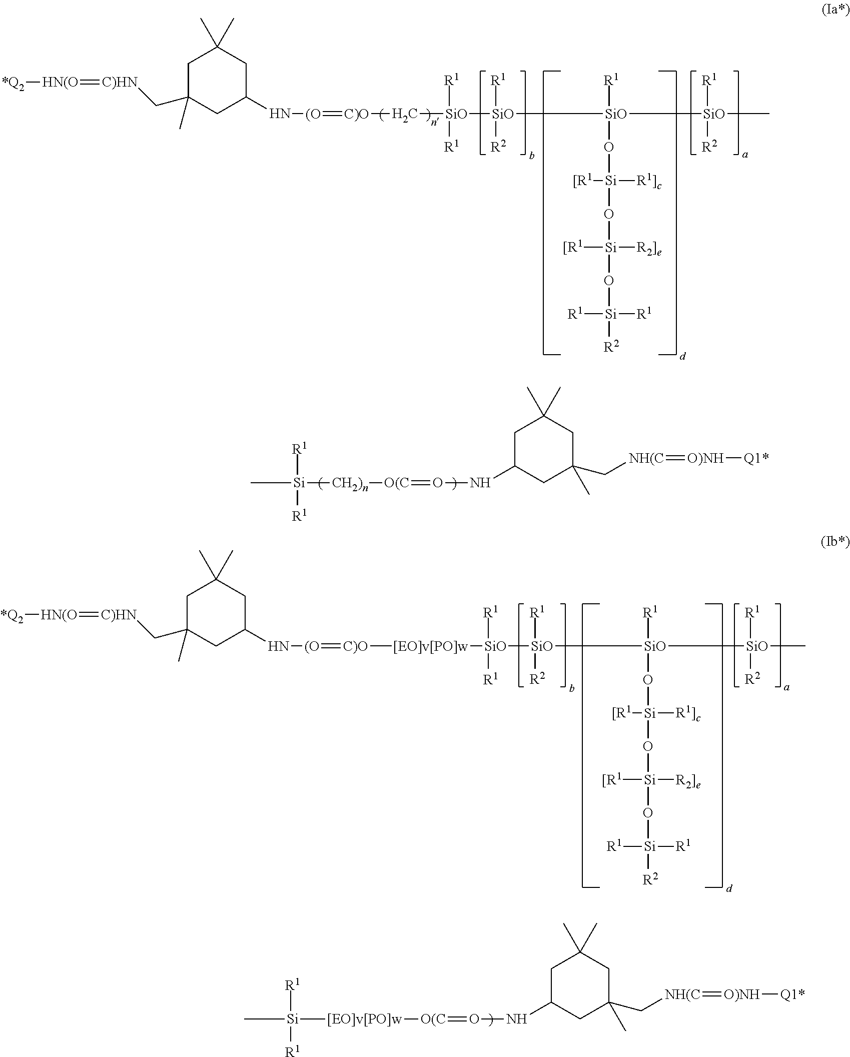

where a is from 1 to 200, in particular where a is from 20 to 100, where b is from 0 to 200, where d is from 0 to 200, where e is from 0 to 200, in each case as defined above, and where R′ in formula Ia* and Ib* are in each case independently identical or different, where R¹ comprises alkyl radicals with 1 to 4 carbon atoms or phenyl radicals, where R² is alkyl radical with 1 to 22 carbon atoms, preferably 1 to 4 carbon atoms, in particular 1, 2, 3, 4 carbon atoms, an alkyl radical with at least one heteroatom selected from N, O, S, such as alkylamine, glycidyloxyalkyl radical, or phenyl radical and where Q1* and Q2* are in each case independently selected from amino acids, amino acid derivatives or salts thereof, where the fragment —NH-Q1* and —NH-Q2*, as a result of reaction of the secondary alpha-amino groups of the amino acids, their derivatives or of the salts with isocyanate, forms the urea group, and where the amino acids comprise nonpolar amino acids alanine, valine, methionine, leucine, isoleucine, proline, tryptophan, phenylalanine, basic amino acids comprising lysine, arginine, histidine, polar and neutral amino acids comprising tyrosine, threonine, glutamine, glycine, serine, cysteine, asparagine and/or the acidic amino acids selected from glutamic acid and aspartic acid, and their mono-, dicarboxylic acid esters, amides with primary amino groups of the amino acids, amides of the carboxylic acid groups of the amino acids and/or esters with the primary hydroxyl groups or thioesters of the HS group or salts thereof, and in formula Ib* with [EO]v[PO]w, where v is from 0 to 200 and w is from 0 to 200, in particular in each case independently at least v and/or w is 5 to 100, or at the same time Q1′ and Q2′ are in each case independently of formulae IVa or IVb,

-T-O—(CH₂—CH₂—O—)ₓ—(CH₂—CH(R#)O—)_{y}(SO)—R″ (IVa)

-T-O—(CH₂—CH₂—O—)ₓ—(CH₂—CH(R#)O—)_{y}—R″ (IVb),

where T=bivalent hydrocarbon radical with 2 to 4 carbon atoms, where x=0 to 200, y=0 to 200, preferably as defined above, preferably x and/or y is 5 to 100, where x and y are integers with the proviso that x or y is at least 1, where R# is hydrogen or methyl, R″ is hydrogen or alkylene, —(CH₂)₂—, —(CH(CH₃))CH₂—, methylene, polymethylene or —(CH₂)₃—, in particular —CH₂—CH₂—, in particular where T=-(CH₂)₂— or —(CH₂)₃—.

FIGS. 1 (1a and 1b) to FIG. 5 represent by way of example the siloxane polymers of the formulae Ia, Ib, Ic obtainable by the process according to the invention, without limiting the invention to these examples. FIG. 1a shows a siloxane polymer of the general formula Ia which is obtainable from the reaction with isophorone diisocyanate and an amino acid, where on the part of the amino acid a hydroxyl or amino group can have reacted with isocyanate, the linker is an alkylene with n or n′ in each case independently an integer between 2 and 40. FIG. 1b** shows a specific compound for Q1′ and Q2′ with a polyether as linker. FIG. 2 shows a preferred embodiment of the general formula Ib which is likewise obtainable by a reaction with an isophorone diisocyanate and an amino acid, where the alpha-amino group of the amino acid has been reacted with isocyanate and the second isocyanate group of the isophorone has been reacted with a hydroxyl-functionalized siloxane, for example a hydroxyalkyl-functionalized siloxane. FIG. 3 specifies in formula Ia* the formula Ia in so far as Q2′ and Q1′ are in each case a bivalent alkylene. FIG. 4 shows Ib* with a bivalent polyether—[EO]v[PO]w-, where v and w are as defined above. The siloxane polymers of the formulae I can also be reacted with an aminoalkyl-functionalized siloxane to give a siloxane polymer with two urea groups and amino acid derivatives as terminal groups. FIG. 5 shows a possible isomer when the process takes place according to an alternative route of variant b) via the preparation of amino acid isocyanates by reacting an amino acid derivative with IPDI to give a compound of the formula IX and then performing a reaction with a siloxane derivative of the formula VI (FIG. 5: Ic).

According to a particularly preferred alternative, it is likewise possible to use a siloxane of the general formula XI in the process according to the invention, in particular as explained below. In the preferred process, R¹⁷ is then hydrogen or -Q1′-A-(C═O)-D-Q1″-NCO, -Q2′-A-(C═O)-D-Q2″-NCO or -Q1′-AH, -Q2′-AH.

A siloxane of the general formula XI as siloxane polymer according to the invention, in particular of the general formula I, is likewise obtainable by the process, where R¹⁷ are in each case independently -Q1 and -Q2 for a2 greater than or equal to 1, in particular with a2 greater than or equal to 2.

1) In general, preferably at least one siloxane of the general formula XI is obtainable by the process according to the invention with a) with R¹⁷ as defined for siloxane polymers in a).

2) Likewise, preferably at least one siloxane of the general formula XI can be used in the process with R¹⁷ corresponding to the definition in b) for the general formula XI

Mₐ₁M^{A}ₐ₂M^{B}ₐ₃D_{b1}D^{A}_{b2}D^{B}_{b3}T_{c1}T^{A}_{c2}T^{B}_{c3}Q_{d1} (XI)

where

### M=[R¹⁶₃SiO_{1/2}]

### M^{A}=[R¹⁷R¹⁶₂SiO_{1/2}]

### M^{B}=[R¹⁸R¹⁶₂SiO_{1/2}]

### D=[R¹⁶₂SiO_{2/2}]

### D^{A}=[R¹⁷₁R¹⁶₁SiO_{2/2}]

### D^{B}=[R¹⁸₁R¹⁶₁SiO_{2/2}]

### T=[R¹⁶SiO_{3/2}]

### T^{A}=[R¹⁷SiO_{3/2}]

### T^{B}=[R¹⁸SiO_{3/2]}

### Q=[SiO_{4/2}],

where R¹⁶, independently of one another, are identical or different linear or branched, saturated or unsaturated hydrocarbon radicals with 1 to 30 carbon atoms or else aromatic hydrocarbon radicals with 6 to 30 carbon atoms, preferably methyl or phenyl, in particular methyl,
a) for the siloxane polymer, in particular of the formula I, shown via the formula XI where R¹⁷ is in each case independently -Q1, -Q2 for a siloxane polymer of the general formula I, where the siloxane of the formula XI (without radicals R¹⁷, i.e. M^{A}=[—R¹⁶₂SiO_{1/2}], D^{A}=[—R¹⁶₁SiO_{2/2}], and/or T^{A}=[—SiO_{3/2}]) corresponds to the fragment B of the formula I and the formula XI with R¹⁷ is equivalent to the formula I with Q2-B-Q1.
b) in the process for the preparation of the siloxane polymers: Alternatively, it is also possible to use a siloxane of the formula XI with R¹⁷ in the process for the preparation of at least one siloxane polymer, in particular of the formula I, with R¹⁷ comprising -Q1′-AH, -Q2′-AH, -Q1′-A-(C═O)-D-Q1″-NCO, OCN-Q2″-D-(O═C)-A-Q2′-, hydrogen for Si—H group,
—OH, —OR¹⁶, in particular —OMe, -AH, in one alternative R¹⁷ is particularly preferably a saturated hydrocarbon radical with terminal —OH or —NH₂ group, preferably with 8 to 30, particularly preferably with 8 to 20 carbon atoms, in particular in MA and optionally D^{A} or R¹⁷ is R¹⁸ in M^{B}, D^{B} and/or T^{B},
R¹⁸ independently of one another are identical or different linear or branched, saturated or olefinically unsaturated hydrocarbon radicals with 8 to 30 carbon atoms, for example decyl-, dodecyl, tetradecyl-, hexadecyl-, octadecyl-, in particular hexadecyl- and octadecyl-,
an aromatic hydrocarbon radical with 6 to 40 carbon atoms, an alkylaryl radical with 7 to 40 carbon atoms,
a linear or branched, optionally double-bond-containing aliphatic hydrocarbon radical with 2 to 30 carbon atoms interrupted by one or more heteroatoms (oxygen, NH, NR′ where R′ is an optionally double-bond-containing C1 to C30-alkyl radical, in particular —CH₃),
a linear or branched, optionally double-bond-containing aliphatic hydrocarbon radical with 2 to 30 carbon atoms interrupted by one or more functionalities selected from the group —OH —O—C(O)—, —(O)C—O—, —NH—C(O)—, —(O)C—NH, —(CH₃)N—C(O)—, —(O)C—N(CH₃)—, —S(O₂)—O—,

### —O—S(O₂)—, —S(O₂)—NH—, —NH—S(O₂)—, —S(O₂)—N(CH₃)—, —N(CH₃)—S(O₂)—,

a terminally OH, OR′, NH₂, N(H)R′, N(R′)₂ (where R′ is an optionally double-bond-containing C1 to C30 alkyl radical) functionalized linear or branched optionally double-bond-containing aliphatic or cycloaliphatic hydrocarbon radical with 1 to 30 carbon atoms or
a blockwise or randomly constructed polyether according to (R⁵—O)ₙ—R⁶, where R⁵ is a linear or branched hydrocarbon radical containing 2 to 4 carbon atoms, n is 1 to 100, preferably 2 to 60, and R⁶ is hydrogen, a linear or branched optionally double-bond-containing aliphatic hydrocarbon radical with 1 to 30 carbon atoms, an optionally double-bond-containing cycloaliphatic hydrocarbon radical with 5 to 40 carbon atoms, an aromatic hydrocarbon radical with 6 to 40 carbon atoms, an alkylaryl radical with 7 to 40 carbon atoms,
or a radical —C(O)—R⁷ where R⁷ is a linear or branched optionally double-bond-containing aliphatic hydrocarbon radical with 1 to 30 carbon atoms, an optionally double-bond-containing cycloaliphatic hydrocarbon radical with 5 to 40 carbon atoms, an aromatic hydrocarbon radical with 6 to 40 carbon atoms, an alkylaryl radical with 7 to 40 carbon atoms, particularly preferably in an alternative R¹⁸ is a saturated hydrocarbon radical with terminal —NH₂ groups, preferably with 8 to 30 carbon atoms, particularly preferably with 8 to 20 carbon atoms, where
a1=0-200, preferably 1-60, in particular 0,
a2=0-30, preferably 1-20, in particular 2-10, such as 2, 3, 4, 5, 6, 7, 8, 9, 10,
a3=0-30, preferably 1-20, in particular 0, such as 1, 2, 2, 3, 4, 5, 6, 7, 8, 9, 10, 15, 20,
b1=2 to 5000, preferably 10 to 1000, in particular 10-500, particularly preferably 2 to 100, preferably 10 to 100,
b2=0 to 100, preferably 1 to 30, in particular 1 to 10 or 0,
b3=0 to 100, preferably 0 to 30, in particular 1 to 10 or 0,
c1=0 to 30, preferably 1 to 30,
c2=0 to 30, preferably 0 to 5, in particular 0,
c3=0 to 30, preferably 0 to 5, in particular 0,
d1=0 to 30, preferably 0 to 5, preferably 0,
with the proviso that at least one of the indices selected from a1, a2 and a3 is not 0, in particular with (a2+b2+c2) greater than or equal to 1, preferably a2 is an integer between 2 and 10, preferably 2 to 5, such as 2, 3, 4 or 5, where it is further preferred that b1 is from 10 to 150, preferably 10 to 100. Optionally, additionally al and/or a3 can be an integer between 2 and 10, preferably 2 to 5, such as 2, 3, 4 or 5. With the proviso that (c1+c2+c3) is an integer greater than or equal to 1 if the sum of (a1+a2+a3) is an integer greater than 2. According to one alternative, a1 and/or a3 can also additionally be an integer greater than 1.

Siloxanes having at least one group selected from hydroxy and amino group that are used in the process and preferred according to the invention are characterized by the parameter characterization selected from the group:

a1=0, a2=2, a3=0, b1=10-100, b2=0, b3=0, c1=0, c2=0, c3=0 and d1=0,
a1=0, a2=2, a3=0, b1=10-100, b2=1-30, b3=0, c1=0, c2=0, c3=0 and d1=0;
a1=0, a2=2, a3=0, b1=20-40, b2=1-30, b3=0, c1=0, c2=0, c3=0 and d1=0,
a1=0, a2=2, a3=0, b1=41-90, b2=1-30, b3=0, c1=0, c2=0, c3=0 and d1=0,
a1=0, a2=2, a3=0, b=5-350, b2=0, b3=0, c1=0, c2=0, c3=0 and d1=0,
a1=0, a2=2, a3=0, b1=15-200, b2=0, b3=0, c1=0, c2=0, c3=0 and d1=0,
a1=0, a2=2, a3=0, b1=10-150, b2=0, b3=1 to 5, c1=0, c2=0, c3=0 and d1=0;
a1=0, a2=0, a3=2, b=5-350, b2=0, b3=0, c1=0, c2=0, c3=0 and d1=0,
a1=0, a2=0, a3=2, b1=15-200, b2=0, b3=0, c1=0, c2=0, c3=0 and d1=0,
a1=2, a2=0, a3=2 to 5, b1=10-150, b2=1-30, b3=0, c10, c2 Z 0, c3 Z 0 and d1=0; where (c1+c2+c3) is greater than or equal to 1 to 3
a1=0, a2=2, a3=0, b1=10-150, b2=0, b3=1-2, c1=0, c2=0, c3=0 and d1=0,
a1=0, a2=2, a3=0, b1=51-90, b2=0, b3=1-2, c1=0, c2=0, c3=0 and d1=0,
a1=0, a2=0, a3=2, b1=10-50, b2=0, b3=1-2, c1=0, c2=0, c3=0 and d1=0,
a1=0, a2=1, a3=1, b1=10-150, b2=0, b3=1 to 5, c1=0, c2=0, c3=0 and
d1=0;

The index numbers a, b, c, d, e, f, a1, a2, a3, b1, b2, b3, c1, c2, c3, d1, d2, d3, v, w, n, n′ etc. given in formulae I, II, III, IV, XI and all associated substructures, which are named for example using Arabic letters, and the value ranges of the stated indices are understood to be average values of the possible statistical distribution of the structures actually present and/or their mixtures. This is also true for structural formulae exactly reproduced as such per se, such as for example for formula I, II, III and III, or IIa, IIb, IIIa, IIIb.

Statistical distributions can be blockwise in structure with any desired number of blocks and any desired sequence or be subject to a randomised distribution, they can also have an alternating structure or else form a gradient via the chain, in particular they can also form all mixed forms in which optionally groups of different distributions can follow one another. Specific embodiments can lead to the statistical distributions experiencing limitations due to the embodiment. For all regions which are not affected by the limitation, the statistical distribution is not changed.

Preferably, R¹⁷ in MA and/or DA optionally T^{A} is selected from the two formulae IX1 and IX2 below or comprise a radical of the formulae XIIa or XIIb.

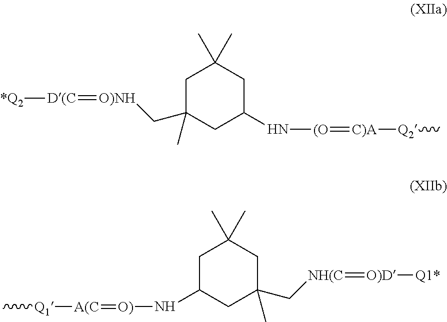

In addition to the amino acid isocyanates of the general formulae (IXa to IXd), it is also possible to react further substituted isocyanate derivatives, preferably from the reaction with diamines comprising a tertiary and a primary amino group on hydrocarbons optionally comprising O or N in the reaction with a reactive hydroxyl- or amino-functional siloxane, for example of the formula VI. The reaction can also take place in a mixture comprising amino acid isocyanates. In formulae IX1 and IX2, Z=-Q1*, Q2* can be derived from formula XIII or an amine, preferably a diamine, such as DMPAPA, etc.

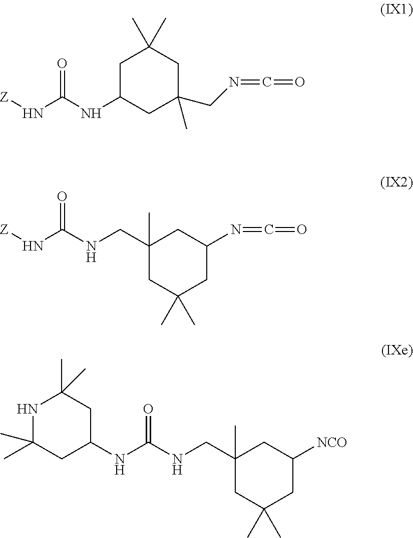

According to one alternative, in addition to the reaction with amino acids, a reaction with sterically hindered amines with a primary amino or hydroxyl group can take place, particular preference being given to the diamines with a sterically hindered nitrogen as basic group, particularly preferably HALS amine of the formula XIII (4-amino-2,2,6,6,-tetramethylpiperidine) or N,N-dimethylaminopropylamine (DMAPA) or 3-(dimethylamino)propylamines (CAS:109-55-7), N-(3-aminopropyl)imidazoles (CAS: 5036-48-6), dimethylethanolamine (CAS:108-01-0), dimethylaminoethoxyethanol (CAS:1704-62-7); trimethylaminoethylethanolamines (CAS: 2212-32-0) or salts thereof. Also conceivable is a reaction of lateral, functional groups or Si—OH groups with one of the aforementioned amines, preferably of the formula XIII.

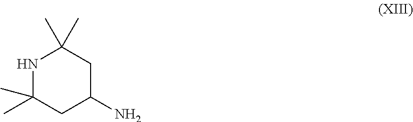

The invention likewise provides a process for the preparation of a siloxane polymer, in particular of the formula I, preferably of the formula XI, as well as siloxane polymers and compositions comprising these siloxane polymers obtainable by the process with a central polysiloxane polymer block B, in particular a process for the preparation of at least one siloxane polymer of the general formula (I), as described above, and also compositions comprising these siloxane polymers or mixtures of the siloxane polymers with a central polysiloxane polymer block B by reacting a) a polysiloxane diisocyanate of the formula VII,

OCN-Q2″-D-(O═C)-A-Q2′-B-Q1′-A-(C═O)-D-Q1″-NCO (VII)

with an amino acid, an amino acid derivative or salt thereof, in particular with a secondary amino-group-having amino acid or a derivative thereof, where the reaction preferably takes place in the molar ratio of at least 1:1 with regard to the isocyanate groups of the polysiloxane to amino or hydroxyl groups of the amino acid or of the amino acid derivative,
and a siloxane polymer of the general formula (I)

Q2-B-Q1 (I)

is obtained, where -Q1 corresponds to the general formula IIa and -Q2 corresponds to the formula IIb,

-Q1=-Q1′-A-(C═O)-D-Q1″-A′-(C═O)-D′-Q1* (IIa)

-Q2=-Q2′-A-(C═O)-D-Q2″-A′-(C═O)-D′-Q2* (IIb)
where A is —NH—, —O— or —S— and D is —NH— in each case independently in formulae IIa and lib,
where A′ is —NH— and D′ is —NH—, —O— or —S— in each case independently in formulae IIa and IIb, where each radical Q1 and Q2 of the formula IIa or IIb has in each case independently at least one bivalent urea group and a further bivalent urea or a carbamate group, or by reacting
   b) a polysiloxane of the formula VI

HA-Q2′-B-Q1′-AH (VI)

with an amino acid isocyanate selected from the formulae IXa, IXb, IXc and IXd

Q2*—NH(CO)NH-″2Q-NCO (IXa)

Q1*—NH(CO)NH-″1Q-NCO (IXb)

Q2*—NH(CO)NH-Q2″-NCO (IXc)

Q1*—NH(CO)NH-Q1″-NCO (IXd)
where A is —NH—, —O— or —S— and D is —NH— in each case independently in formulae VII, I, VI, IXa, IXb, IXc and IXd, in particular comprising IXa and IXb,

with Q1′ and Q2′ in each case independently comprising a bivalent hydrocarbon radical with 6 to 200 carbon atoms optionally comprising at least one heteroatom comprising O, N or S, a bivalent radical comprising aryl, arylalkyl groups or a bivalent radical comprising aryl, arylalkyl groups optionally comprising at least one heteroatom O, N or S or polyether radicals containing alkyl, aryl or alkyl and aryl groups, in each case independently in formulae VII, I and/or VI,
with Q1″ and Q2″ in each case independently comprising a bivalent linear, branched and/or cyclic alkyl radical with 4 to 200 carbon atoms, in particular a cyclic C6 alkyl radical with alkyl side chains, or a bivalent radical comprising an aryl and/or arylalkyl radical with 6 to 200 carbon atoms, in each case independently in formulae VII, I, IVa and/or IVb,
with —NH-Q1* and —NH-Q2* comprising in each case independently radicals which are derived from an amino acid, amino acid derivative or salt thereof. Optionally in each case independently with —O-Q1* and —O-Q2*, —S-Q1* and —S-Q2* comprising in each case independently radicals which from an amino acid, amino acid

The reaction in step a) can proceed in the presence of a catalyst, such as the catalysts known in the prior art for polyurethane production and isocyanate trimerization. By way of example, mention is made of tertiary amines such as triethylamine, tetraethylenediamine, or strong bases such as DBU, and also tin and bismuth compounds, such as, for example, dibutyltin laurate or tin(II) octoate.

According to one process variant, the formulae IVa, IVb and I comprise as —NH-Q1* and —NH-Q2* in each case independently radicals which are derived from an amino acid, amino acid derivative or salt thereof, and —NH in —NH-Q1* and —NH-Q2* is in each case in the alpha position relative to an ester or a carboxy group of the amino acid, the amino acid derivative or salt thereof, where the ester comprises alkyl esters with 1 to 25 carbon atoms or aryl esters. Preference is given to methyl, ethyl or phenyl esters. HD′-Q1*,

HD′-Q2* with HD′ in each case H₂N— and Q1* and Q2* amino acid radical.

The process according to the invention can in particular comprise the following steps and comprise individual steps:

(I) H—B—H (x)+Q1′-AH (VIIIa), Q2′-AH (VIIIb)→HA-Q2′-B-Q1′-AH (VI)
(II) HA-Q2′-B-Q1′-AH+diisocyanate→OCN-Q2″-D-(O═C)-A-Q2′-B-Q1′-A-(C═O)-D-Q1″-NCO (VII)
(III) OCN-Q2″-D-′(O═C)-A-Q2′-B-Q1′-A-(C═O)-D-Q1″-NCO (VII)+amino acid→Q2-B-Q1 (I)
or alternatively
(Ia) diisocyanate (e.g. IPDI)+amino acid→Q2*—NH(CO)NH-″2Q-NCO (IXa)+Q1*—NH(CO)NH-″1Q-NCO (IXb) and optionally Q2*—NH(CO)NH-Q2″-NCO (IXc) and/or Q1*—NH(CO)NH-Q1″-NCO (IXd)
(Ib) H—B—H (x)+Q1′-AH (VIIIa), Q2′-AH (VIIIb) HA-Q2‘-B-QI’-AH (VI)

### (II) HA-Q2′-B-Q1′-AH (VI)+Q2*—NH(CO)NH-″2Q2-NCO (IXa)/Q1*—NH(CO)NH-″1Q-NCO (IXb)→Q2-B-Q1 (I)

To prepare the siloxane polymers, firstly

(i) a polysiloxane-group-containing linear and/or branched polymer block B, in particular of the formulae IIIa and/or IIIb or of the formula XI with R¹⁷=H and a2 is greater than or equal to 2, b1 is greater than or equal to 1 or a1 is greater than or equal to 1, a2 is greater than or equal to 1 and b1 is greater than or equal to 1, with at least two terminal Si—H groups or at least one terminal Si—H group and at least one lateral Si—H group, e.g. H—B—H(X), where —H corresponds to two Si—H groups, are reacted
(ii) with an olefinic compound comprising alkylene and optionally at least one heteroatom such as N, O, S, in particular alkenylenol, alkylenamine, alkylencarboxylic acid, alkylene ester, alkylenamide or an olefinic polyether, where the olefinic compound in each case independently has an allyl or vinyl group and corresponds to the formulae VIIIa and/or VIIIb

Q1′-AH (VIIIa)

Q2′-AH (VIIIb)

with Q1′ and Q2′ in each case independently comprising an alkenylene with 6 to 200 carbon atoms optionally comprising at least one heteroatom O or N, aryl or arylalkyl groups optionally comprising at least one heteroatom O or N, olefinic polyether with -AH in formulae VIIIa and VIIIb independently selected from —OH or —NH₂. The reaction preferably takes place in (iii) in the presence of a catalyst, such as a Karstedt catalyst, to give a polysiloxane of the formula VI,

HA-Q2′-B-Q1′-AH (VI)

where in each case independently in formulae VIIIa, VIIIb and VI where AH are independently selected from —OH and —NH₂, and with -Q2′- and -Q1′- in each case independently comprising a bivalent hydrocarbon radical with 6 to 200 carbon atoms optionally comprising at least one heteroatom O or N, a bivalent radical comprising aryl, arylalkyl groups optionally comprising at least one heteroatom O or N or olefinic polyether.

For the reaction of olefinic compounds with the Si—H group, hydrosilylation catalysts are used. The use of a Karstedt catalyst is customary. Generally, preference is given to platinum catalysts in which platinum(0) is present.

Mercaptoalkyl substituted siloxanes, in particular of the formula I, VI or XI can be prepared by the person skilled in the art by processes known to him from the prior art via a condensation and/or equilibration.

In a subsequent process step, the polysiloxane of the formula VI

HA-Q2′-B-Q1′-AH (VI)

with A selected from −O, —NH or AH selected from —OH, —NH₂, and —SH with -Q2′- and -Q1′- as defined above, can be reacted with a diisocyanate to give a polysiloxane diisocyanate of the formula VII, preference being given to the diisocyanate IPDI,

OCN-Q2″-D-(O═C)-A-Q2′-B-Q1′-A-(C═O)-D-Q1″-NCO (VII)

with -Q2″- and/or -Q1″- independently selected from a bivalent, linear, branched and/or cyclic alkyl radical with 4 to 200 carbon atoms, in particular an isophorone radical, or a bivalent radical comprising an aryl and/or arylalkyl radical with 6 to 200 carbon atoms, where the molar ratio of HA groups in the polysiloxane to isocyanate groups is at least 1:1, in particular the ratio is 1:100 to 1:1, preferably 1:10 to 1:1.

Particular preference is given to reacting a diisocyanate with an amino acid derivative or salt thereof, such as the hydrochlorides of methyl or ethyl esters of alpha-amino acids with amino group in the alpha position, to give an amino acid isocyanate, in particular to give an amino acid isocyanate selected from the formulae IXa and IXb.

In the next process step, the prepared polysiloxane diisocyanate of the formula VII or any desired polysiloxane diisocyanate of the formula VII prepared by another process

OCN-Q2″-D-(O═C)-A-Q2′-B-Q1′-A-(C═O)-D-Q1″-NCO (VII)

where B is a linear and/or branched polysiloxane polymer block B, with -Q2′- and -Q1′- in each case independently comprising a bivalent hydrocarbon radical with 6 to 200 carbon atoms optionally comprising at least one heteroatom O, N or S, a bivalent radical comprising aryl, arylalkyl groups optionally comprising at least one heteroatom O, N, or S, polyether radicals containing alkyl, aryl or alkyl and aryl groups, where A is in each case independently —NH—, —O— or —S— and D is —NH— in each case independently in formula VII, and with -Q2″- and/or -Q1″- independently selected from a bivalent, linear, branched and/or cyclic alkyl radical with 4 to 200 carbon atoms, or a bivalent radical comprising an aryl and/or arylalkyl radical with 6 to 200 carbon atoms, can be reacted with an amino acid, amino acid derivatives or salts thereof, in particular with a secondary amino group of the amino acid or of the amino acid derivative, preferably with a methyl or ethyl ester of an alpha-amino acid or a salt of the specified compounds.

FIG. 6 shows an obtainable diisocyanate according to formula VII with D in each case —NH—. In the process according to the invention, difunctional isocyanates selected from the group comprising for example toluene 2,4-diisocyanate (TDI), diphenylmethane diisocyanate or methylenediphenyl diisocyanate (MDI), hexamethylene diisocyanate (HMDI), 2,2,4-trimethylhexane 1,6-diisocyanate (TMDI), polymeric diphenylmethane diisocyanate (PMDI), isophorone diisocyanate (IPDI), 4,4′-diisocyanatodicyclohexylmethane (H12MDI) can be used, with the aliphatic products being preferred, and isophorone diisocyanate (IPDI) being particularly preferred.

Some of these isocyanates have stereocentres. In particular reference is made to the isomers of isophorone. All conceivable isomers are expressly incorporated in the scope of this invention. Thus, for example, isophorone diisocyanate can be differentiated into a cis and a trans isomer. Particular preference is given to an isophorone diisocyanate of a cis/trans mixture of 5:1 to 1:5, preferably 3:1 to 1:3, further preferably 1:1. A particularly preferred commercial product consists of a cis/trans mixture of 3:1. The use of commercial isophorone diisocyanate is preferred. Isophorone diisocyanate is obtainable under other names which are included as synonyms in the scope of this invention: 3-isocyanatomethyl-3,5,5-trimethylcyclohexyl isocyanate, 5-isocyanato-1-(isocyanatomethyl)-1,3,3-trimethylcyclohexane, CA RN: 4098-71-9. Various trade names are customary; they often contain the name of the parent molecule isophorone, although other trade names are also customary: e.g. Desmodur®I (BAYER), Isocur IPDI 22-200 (ISO-ELEKTRA), VESTANAT® IPDI (EVONIK INDUSTRIES), which are likewise incorporated within the scope of the present invention. Customary specifications for isophorone diisocyanate are: total chlorine content <400 mg/kg, hydrolysable chlorine <200 mg/kg, purity >99.5% by weight, refractive index n25D 1.483 (DIN 51 423, part 2), NCO content 37.5-37.8% by weight (EN ISO 11 909/ASTM D 2572), the commercial product is described as colourless to light yellow. The specified isocyanates can optionally at least partially comprise prepolymers.

Suitable isocyanate-group-containing compounds are all known isocyanates. Within the context of the teaching according to the invention, preference is given to e.g. aromatic, aliphatic and cycloaliphatic polyisocyanates with a number-average molar mass of less than 800 g/mol. Thus, of suitability for example are diisocyanates selected from the series 2,4-/2,6-toluene diisocyanate (TDI), methyldiphenyl diisocyanate (MDI), triisocyanatononane (TIN), naphthyl diisocyanate (NDI), 4,4′-diisocyanatodicyclohexylmethane, 3-isocyanatomethyl-3,3,5-trimethylcyclohexyl isocyanate (isophorone diisocyanate=IPDI), tetramethylene diisocyanate, hexamethylene diisocyanate (HDI), 2-methylpentamethylene diisocyanate, 2,2,4-trimethyl-hexamethylene diisocyanate (THDI), dodecamethylene diisocyanate, 1,4-diisocyanatocyclohexane, 4,4′-diisocyanato-3,3′-dimethyldicyclohexylmethane, 4,4′-diisocyanatodicyclohexylpropane(2,2), 3-isocyanatomethyl-1-methyl-1-isocyanatocyclohexane (MCI), 1,3-diisooctylcyanato-4-methyl-cydohexane, 1,3-diisocyanato-2-methylcyclohexane and [alpha],[alpha],[alpha]′,[alpha]′-tetramethyl-m- or -p-xylylene diisocyanate (TMXDI), and mixtures consisting of these compounds.

Preferred starting materials for the preparation of the urethane groups and preferably of the urea-group-containing compounds are isophorone diisocyanate (IPDI) and/or 4,4′-diisocyanatodicyclohexylmethane.

Preferably, the following amino acids or amino acid derivative with a secondary amino group are used in the process, and in particular the secondary amino acid group is reacted with an isocyanate group.

The amino acid, the amino acid derivative or salt is preferably selected from the following amino acids, amino acid derivatives or salts thereof comprising nonpolar amino acids selected from alanine, valine, methionine, leucine, isoleucine, proline, tryptophan, phenylalanine, basic amino acids comprising lysine, arginine, histidine, polar and neutral amino acids comprising tyrosine, threonine, glutamine, glycine, serine, cysteine, asparagine and/or acidic amino acids selected from glutamic acid and aspartic acid, where the derivatives of the amino acids comprise the mono-, dicarboxylic acid esters, amides of the primary amino groups of the amino acids, amides of the carboxylic acid groups of the amino acids and/or esters with the primary hydroxyl groups or thioesters of the HS group of the amino acids, in particular the alkyl esters of the amino acids, preferably the methyl, ethyl, phenyl esters of the amino acids or salts thereof. It is likewise preferably possible to use esters with 1 to 15 carbon atoms, hydroxycarboxylic acid esters, fruit acid esters, fatty acid esters of the amino acids.

The invention also provides compositions obtainable by the process according to the invention, in particular comprising siloxane polymers with at least two urea and two carbamate groups and comprising an amino acid radical which is preferably bonded to the siloxane polymer via the alpha-position amino groups. The amino acid radical can be a derivative of an amino acid or a salt. The salt is preferably a physiologically compatible salt, comprising hydrochlorides, aspartic acid, fruit acids, generally hydroxyl acids, mineral acid salts, and further pharmacologically compatible salts known to the person skilled in the art, especially of carboxylic acids.

According to a further embodiment, the invention provides a composition comprising siloxane polymers with a central polysiloxane polymer block B and mixtures comprising

(i) at least one siloxane polymer of the general formula I, and mixtures comprising this polymer,
(ii) at least one siloxane polymer of the general formula Ia, and mixtures comprising this polymer or
(iii) at least one siloxane polymer of the general formula Ib, and mixtures comprising this polymer, and of the formula Ia* and/or Ib* and also mixtures thereof or mixtures comprising these.

The invention further provides an intermediate for the preparation of siloxane polymers, in particular of the formula I, selected from amino acid isocyanates selected from the formulae IXa, IXb, IXc and IXd, in particular IXa*, IXb*, and optionally IXc* and IXd*, or salts thereof or mixtures of the amino acid derivatives.

Q2*—NH(CO)NH-2Q-NCO (IXa)

Q1*—NH(CO)NH-1Q-NCO (IXb)

Q2*—NH(CO)NH-Q2″-NCO (IXc)

Q1*—NH(CO)NH-Q1″-NCO (IXd)

Where -″2Q- and -″1Q- mean that the secondary isocyanate groups have reacted with the amino acid and the primary —CH₂—NCO group can later react with a polysiloxane of the general formula (VI) HA-Q2′-B-Q1′-AH, in particular where B is formula IIIa or IIIb, and alternatively in the meaning of the formula XI where R¹⁷ is in each case independently -Q1′-AH or HA-Q2′- can be reacted.

With Q2*, Q2″, Q1* and Q1″, as defined above, in particular with amino acid isocyanates of the formulae IXa* and IXb* or salts thereof

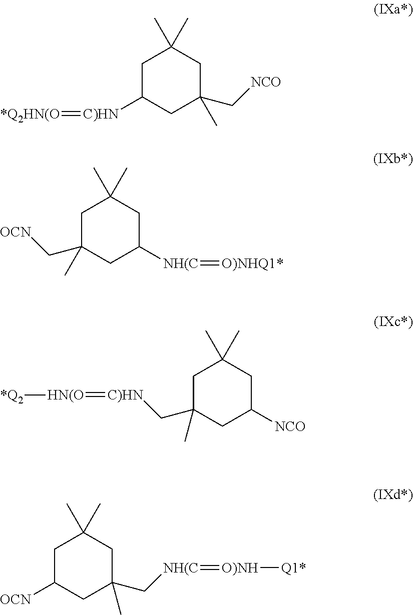

The siloxane polymers of the invention according to the invention have advantageous properties since they advantageously influence the combability of keratin fibres, in particular of hair. In particular, the force which has to be applied for combing can be considerably reduced both compared with untreated hair and also compared with wet hair treated with a standard formulation. The siloxanes are rinsed out of the treated hair beforehand for one minute. The reduction in the force to be applied for combing a defined hair sample is then measured. The siloxanes according to the invention reduce the force by about 51 to 55%, with the siloxane where n=80, IPDI+methyltryptophan giving the best results. The matrix formulation comprises 0.5% by weight of ceterareth-25, 5.0% by weight of cetyl alcohol, 1.0% by weight of cetrimonium chloride, ad 100% by weight of water, the pH is about 4.3. The formulations according to the invention moreover comprise 0.5% by weight of siloxane and ad 100% by weight of water. Similarly, the siloxane where n=80+IPDI+histidine, and the siloxane where n=30, +IPDI+methyltryptophan exhibit a reduction in the combing force by 50%. The pH can be regulated with citric acid.

Consequently, the invention provides a formulation comprising at least one siloxane polymer or a mixture comprising at least one siloxane polymer or siloxane polymer prepared via the intermediate and at least one auxiliary. Preferably, the formulation is a cosmetic rinse for hair, care skin or hair product, lacquer, hair spray, hair colorants, colour, mouthwash, pharmaceutical formulation, impression material (technical, pharmaceutical, cosmetic, dental), cleaner, woodcare product, paint care.

The invention further provides the use of the siloxane polymers, of the obtainable compositions comprising siloxane polymers by the process according to the invention as additive in cosmetic formulations, as additive in pharmaceutical formulations, in paints, pastes, as foam stabilizer or foam additive for polyurethane foams, in particular polyurethane rigid foams and polyurethane flexible foams, as hand improvers or impregnating agents during the production of fibres, textiles, in cosmetic formulations for the treatment, post-treatment and protection of keratin fibres, in particular in hair conditioning formulations, and skin and skin appendages, as additive in detergents, fabric softener formulations, in cosmetic formulations including creams, rinses, hair washing compositions, washing compositions, setting agents, care rinses, care pastes, sprays, hairsprays, for improving the combability of keratin or textile fibres of natural or synthetic origin.

The present invention further provides the use of the siloxanes according to the invention and/or of the siloxanes obtainable by the process according to the invention for producing formulations, in particular of care and cleaning formulations for use in the domestic and industrial sector. Preferred care and cleaning formulations for use in the domestic and industrial sector are in this connection textile care compositions, such as for example fabric softeners, and care compositions for hard surfaces.

The general synthesis of an isocyanate-terminated PDMS takes place by reacting hydroxy-terminated PDMS of different chain lengths, such as (n=30 or 80) (The specific compounds are illustrated in more detail in the preparation examples; 14 and 15) with isophorone diisocyanate (16), which is particularly recommended on account of its differently reactive isocyanate groups for a functionalizaton of the PDMS. In this way, it is possible to exclude the diisocyanate component from reacting twice with the hydroxyl groups of the PDMS and there being no free isocyanate group available for a further reaction with a substituent. Furthermore, gelation of the reaction mixture caused by the formation of high molecular masses can be prevented. For this, the isophorone diisocyanate (16) is firstly introduced into a secured apparatus and reacted without dilution, with the addition of catalytic amounts of triethylamine, with the α,ω-bis(hydroxyhexyl)polydimethylsiloxane (PDMS-30 or PDMS-80) (14 or 15) added dropwise.

The suitability of the short-chain valine-terminated PDMS 20 for use in conditioning rinses has been assessed in accordance with the application tests described above. PDMS-30-Val (20) accordingly has a considerably positive effect, both with the addition of an ammonium chloride and also in pure form, on the detangling, the combability and the wet feel of the hair samples following their treatment. The assessment was carried out by a trained panel of 4 persons by reference to wet feel samples using the grades 1, 2, 3, 4 and 5, with 5 indicating the best sensory properties, i.e. a very smooth feel, and, in contrast to this, the value 1 conveying a harsh or rough feel.

However, since valine is more of a nonpolar amino acid, the functionalization of the PDMS via the indicated diisocyanate is used on an amino acid which has a polar character, such as tryptophan.

The tryptophan-PDMS derivatives 23 and 24 prepared by means of a diisocyanate, and the long-chain valine-PDMS derivative 21 are likewise subjected to first application tests by a known method for the purposes of assessing their suitability in hair care products. Here, the force is measured which is required for combing a hair sample, and can be given in the form of the improved combability of the preparation. The products 21, 23 and 24 were applied to the hair sample with the addition of an ammonium chloride compound in a fixed test formulation and thoroughly rinsed again after a certain time. All of the hair samples treated with the prepared PDMS-amino acid derivatives exhibit a good reduction in the combing forces, with the derivatives having a more basic character, such as PDMS-30-Trp (23) and PDMS-80-Trp (24), bringing about a slightly improved combability of the preparations compared to PDMS-80-Val (21).

The invention likewise provides processes for the purification of the siloxane polymers, by dissolving the siloxane polymers in an alcoholic solution or in alcohol and precipitating them in an aqueous phase or in water. The purified siloxane polymers can be dried in vacuo.

The invention likewise provides processes for the purification of the siloxane polymers, by washing the polysiloxanes as crude product in a mixture, in particular comprising dichloromethane and dimethylformamide, with dist. water and optionally sat. aqueous sodium hydrogen carbonate solution. The organic phases of the mixture are combined, dried over a drying agent and the solvent is removed from the mixture in vacuo. The resulting crude product is dissolved in alcohol, in particular ethanol, and precipitated in dist. water and optionally centrifuged. The drying can take place in vacuo, preferably in high vacuum.

Wherever reference is made within the scope of this invention to natural substances, e.g amino acid, in principle all isomers are intended, preference being given to the naturally occurring isomers in each case, in the case specified here thus the alpha-amino acids. As regards the definition of natural substances, reference is made to the scope of the “Dictionary of Natural Products”, Chapman and Hall/CRC Press, Taylor and Francis Group, e.g. in the online version from 2011: http://dnp.chemnetbase.com/.

Wherever molecules or molecule fragments have one or more stereocentres or can be differentiated into isomers on account of symmetries or can be differentiated into isomers on account of other effects e.g. restricted rotation, all possible isomers are included by the present invention. Isomers are known to the person skilled in the art, reference being made in particular to the definitions by Prof. Kazmaier of the University of Saarland, e.g. http://www.uni-saarland.de/fak8/kazmaier/PDF_files/vorlesungen/Stereochemie %2Strassb %20Vorlage.pdf. In particular, all options which arise from the stereochemical definitions of tacticity are included, e.g. isotactic, syndiotactic, heterotactic, hemiisotactic, atactic. Within the context of the invention, preference is given to polyethers and polyether fragments with at least partial atactic substituent sequence.

### BRIEF DESCRIPTION OF THE DRAWINGS

FIGS. 1 (1a and 1b) to FIG. 5 represent by way of example the siloxane polymers of the formulae Ia, Ib, Ic obtainable by the process according to the invention, without limiting the invention to these examples.

FIG. 1a shows a siloxane polymer of the general formula Ia which is obtainable from the reaction with isophorone diisocyanate and an amino acid, where on the part of the amino acid a hydroxyl or amino group can have reacted with isocyanate, the linker is an alkylene with n or n′ in each case independently an integer between 2 and 40.

FIG. 1b shows a specific compound for Q1′ and Q2′ with a polyether as linker.

FIG. 2 shows a preferred embodiment of the general formula Ib which is likewise obtainable by a reaction with an isophorone diisocyanate and an amino acid, where the alpha-amino group of the amino acid has been reacted with isocyanate and the second isocyanate group of the isophorone has been reacted with a hydroxyl-functionalized siloxane, for example a hydroxyalkyl-functionalized siloxane.

FIG. 3 specifies in formula Ia* the formula Ia in so far as Q2′ and Q1′ are in each case a bivalent alkylene.

FIG. 4 shows Ib* with a bivalent polyether -[EO]v[PO]w-, where v and w are as defined above. The siloxane polymers of the formulae I can also be reacted with an aminoalkyl-functionalized siloxane to give a siloxane polymer with two urea groups and amino acid derivatives as terminal groups.

FIG. 5 shows a possible isomer when the process takes place according to an alternative route of variant b) via the preparation of amino acid isocyanates by reacting an amino acid derivative with IPDI to give a compound of the formula IX and then performing a reaction with a siloxane derivative of the formula VI (FIG. 5: Ic).

FIG. 6 shows an obtainable diisocyanate according to formula VII with D in each case —NH—.

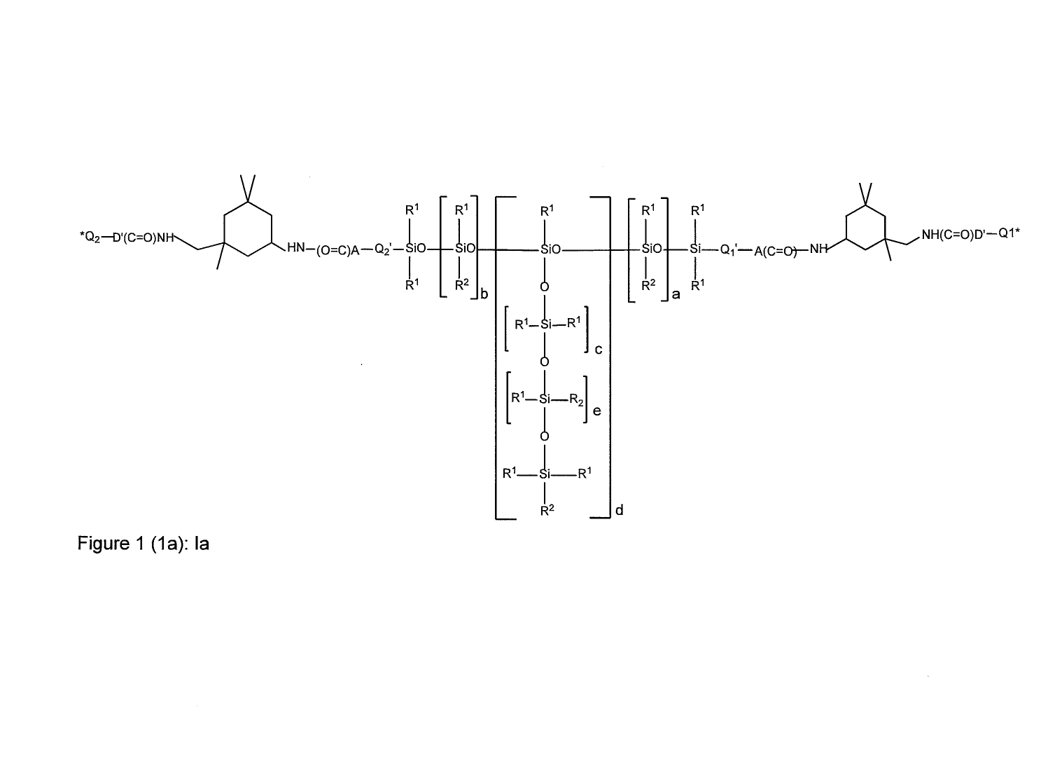

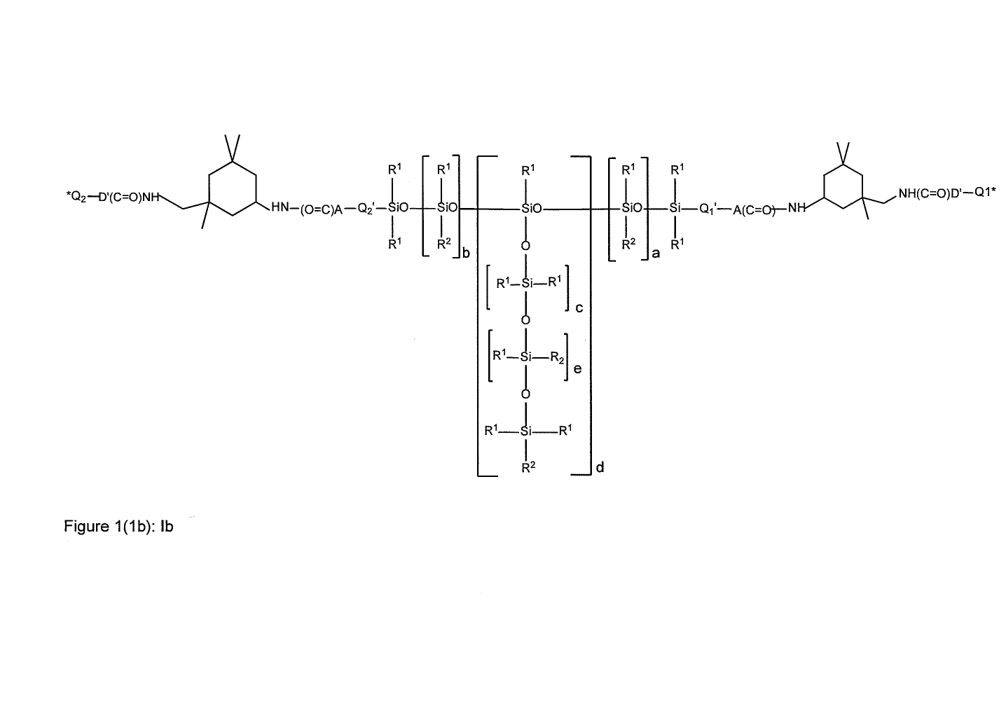

The following examples illustrate the siloxane polymers according to the invention and also the process according to the invention in more detail without limiting the invention to these examples.

### OPERATIVE EXAMPLES

The subject matter of the present invention is elucidated in more detail below, without any intention that the subject matter of the invention should be confined to these exemplary embodiments.

Analysis:

MALDI-TOF-MS: Ultraflex time of flight-mass spectrometer, Bruker. 337 nm nitrogen laser, linear mode or reflector mode. Weighed samples were dissolved in a suitable solvent. The matrix used was dithranol (DIT) or 2,5-dihydroxybenzoic acid (DHB).

FT-IR-spectra: FT-IR-5SXB, Nicolet used. Calibration: by means of HeNe-laser. ATR measurements: specac golden-gate diamond ATR unit.

NMR spectroscopy: 300 MHz-NMR spectrometer, Bruker, model Avance III -300, magnetic field strength 7.05 Tesla. Absorption frequency: ¹H-NMR at 300 MHz, 13C{1H}-NMR at 75 MHz. 200 or 500 MHz-NMR spectra: FT-NMR spectrometer, Bruker DRX200 or DRX500.

### Example 1

### Synthesis of Isocyanate-Terminated Polydimethylsiloxanes

The synthesis of an isocyanate-terminated PDMS was carried out by reacting hydroxy-terminated PDMS of different chain lengths (n=30 or 80) (14 or 15) with isophorone diisocyanate (16). The advantage of the IPDI is that the diisocyanate component does not react twice with the hydroxyl groups of the PDMS. Furthermore, it was possible to avoid a gelation of the reaction mixture caused by the formation of high molecular masses. Isophorone diisocyanate (16) is placed in a secured apparatus and catalytic amounts of triethylamine are added with the dropwise-added α,ω-bis(hydroxyhexyl)polydimethylsiloxane (PDMS-30, n=30 or PDMS-80, n=80) (14 or

15) reacted without dilution. The reaction of the α,ω-bis(hydroxyhexyl)polydimethylsiloxanes 14 and 15 with isophorone diisocyanate (16) to give the α,ω-bis[hexyl(3-(isocyanatomethyl)-3,5,5-trimethylcyclohexyl)carbamyl]polydimethylsiloxanes 17 and 18 (PDMS-30-IPDI, n=30 or PDMS-80-IPDI, n=80) can preferably take place in dichloromethane (60° C., 2 h).

Analysis of isocyanate-terminated PDMS: MS, ¹H-NMR-, IR spectroscopy, molar masses (MALDI-TOF) confirm compounds 17 and 18; IR: characteristic C—H stretching and deformation vibrations of the polydimethylsiloxane at 2961, 1412 and 1257 cm−1, 2256 cm−1 free isocyanate groups, 1709 cm⁻¹ C═O stretching vibration urethane unit.

### Example 2

### Synthesis of Valine-Terminated Polydimethylsiloxanes

Valine-terminated polydimethylsiloxane is prepared by reacting α,ω-bis[hexyl(3-(isocyanatomethyl)-3,5,5-trimethylcyclohexyl)carbamyl]polydimethylsiloxane 17 or 18 in dichloromethane (CH₂Cl₂) with the formation of the valine-terminated polydimethylsiloxanes 20 and 21 (PDMS-30-Val and PDMS-80-Val). The starting materials are completely soluble in dichloromethane (CH₂Cl₂). The products are obtained by extractive separation from the organic phase, dried and analysed.

IR spectra of the valine-terminated PDMS (PDMS-30-Val and PDMS-80-Val) (20 and 21): Molecular vibrations PDMS, C═O: Urethane group at 1726 and 1729 cm⁻¹, C═O: 1635 and 1641 cm⁻¹ urea group, missing: NCO band at 2256 cm⁻¹.

MS measurements (MALDI-TOF): (m/z=1433 for 20 and m/z=3507 for 21).

Methyl ester protective groups of the valine substituents are detected only in traces. 1H-NMR spectroscopy: shows the formation of valine-terminated PDMS derivatives 20 (n=30) and 21 (n=80).

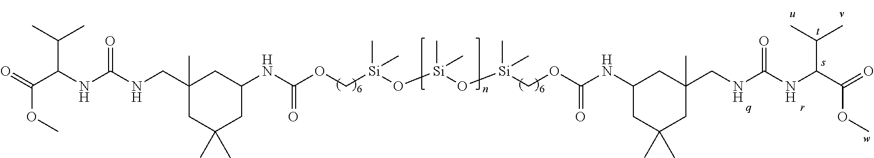

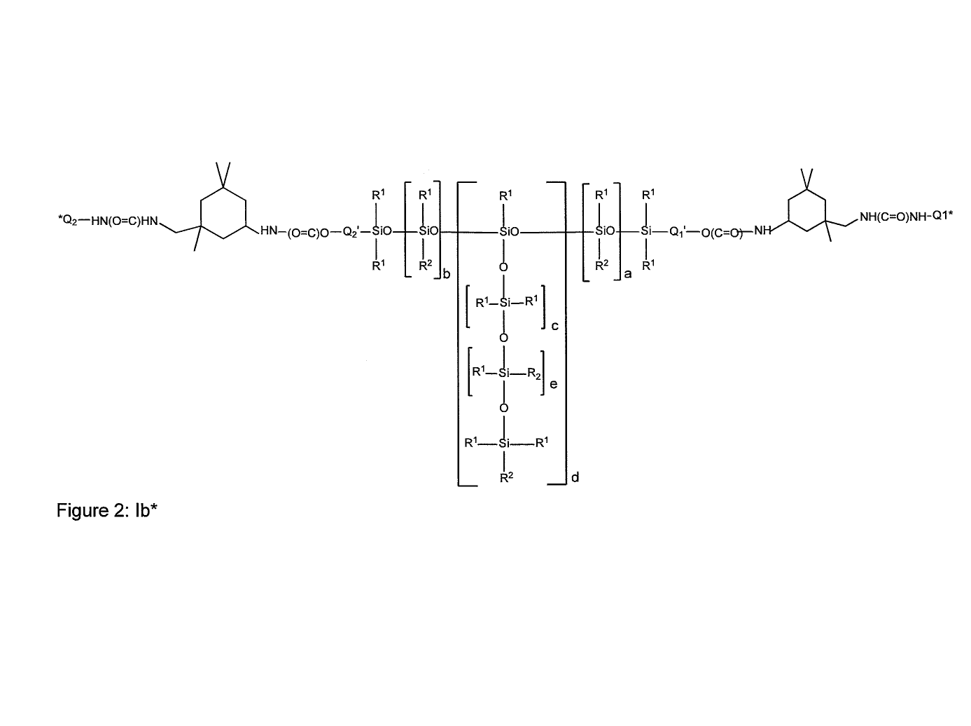

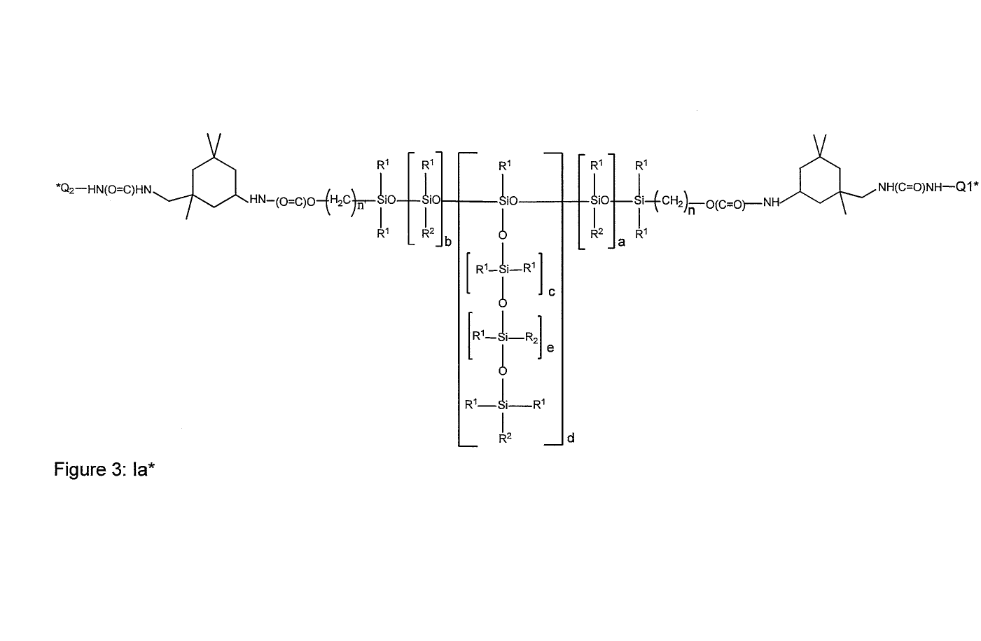

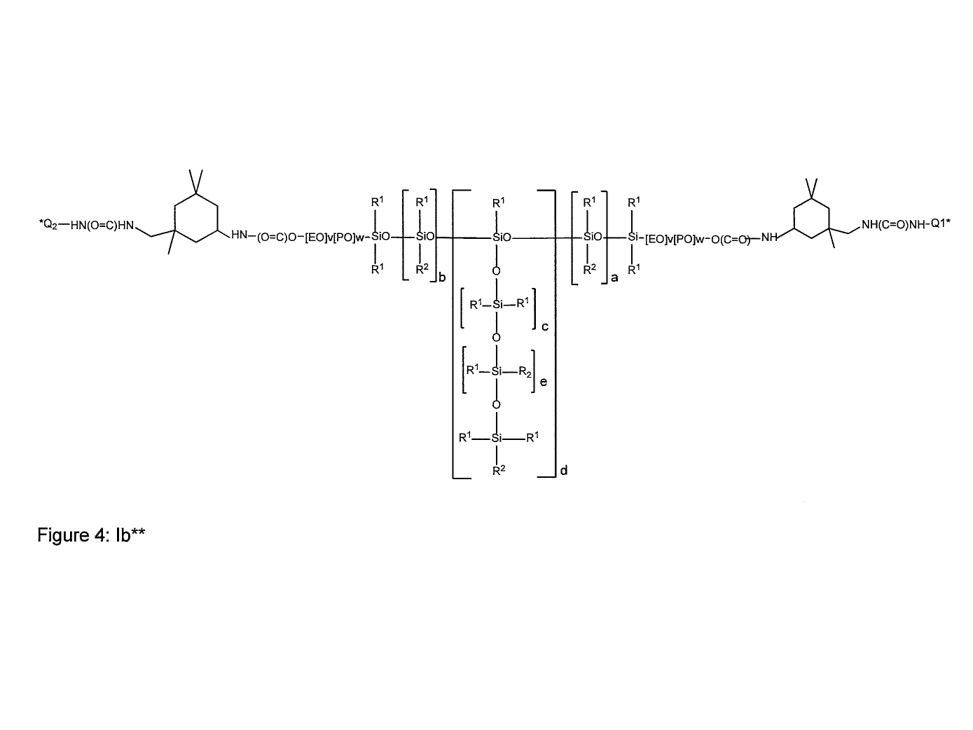

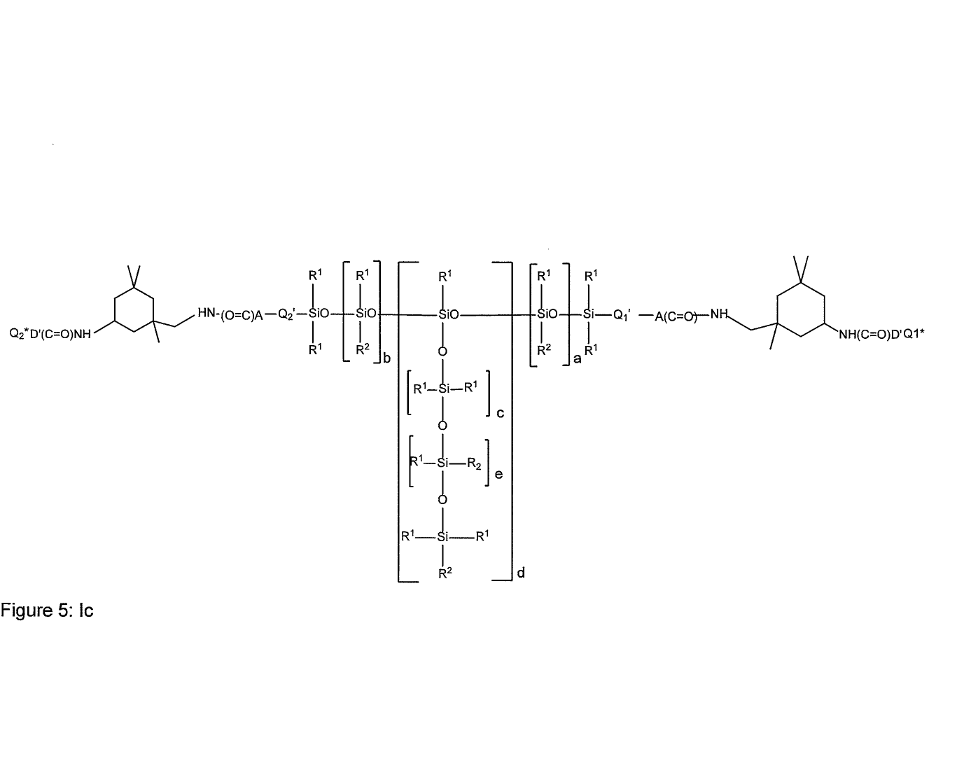

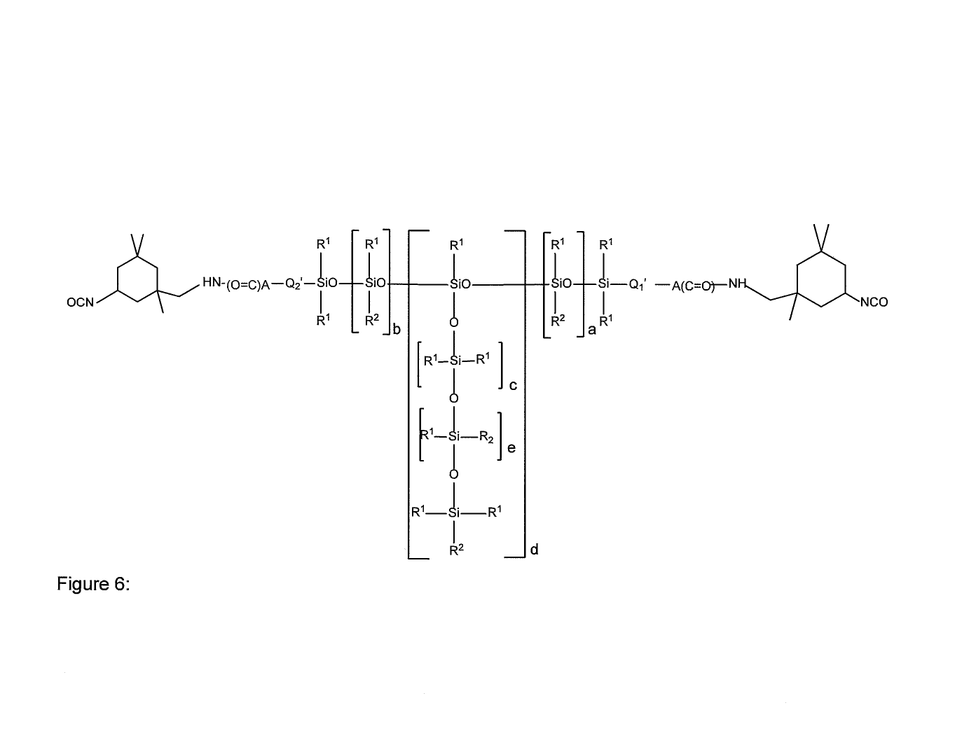

FIG. 2 Valine-terminated polydimethylsiloxane PDMS-30-Val (20), n=30 or PDMS-80-Val (21), n=80, (¹H-NMR: with a to p as Ex. 4)

### Example 3

### Synthesis of Tryptophan-Terminated Polydimethylsiloxanes

Tryptophan-terminated PDMS is prepared by reacting isocyanate-modified PDMS of different chain lengths in solvent mixture of dichloromethane and DMF (1:1). The crude product is reprecipitated in aqueous solution. Solvent residues of the DMF are thus removed from the products. Tryptophan-terminated PDMS are dried under high vacuum.

IR and ¹H-NMR spectroscopy, MALDI-TOF spectrometry confirm the formation of the tryptophan-terminated polydimethylsiloxane.

Freely present isocyanate end groups can be avoided by targeted hydrolysis. The achieved purity of the PDMS derivatives permits their use in cosmetic formulations for the skin.

The preparation of the products 23 and 24 can be described as follows: Reaction of PDMS-30-IPDI (17, n=30) and PDMS-80-IPDI (18, n=80) with tryptophan methyl ester hydrochloride (22) for example in the presence of dichloromethane, triethylenamine, at room temperature for about 24 hours to give tryptophan-terminated polydimethylsiloxanes 23 and 24 (PDMS-30-Trp and PDMS-80-Trp) optional removal of the hydrochloride of the triethylenamine.

### Example 4

### Synthesis of Short-Chain α,ω-bis[hexyl(3-(isocyanatomethyl)-3,5,5-trimethylcyclohexyl)carbamyl]polydimethylsiloxane (PDMS-30-IPDI) (17)

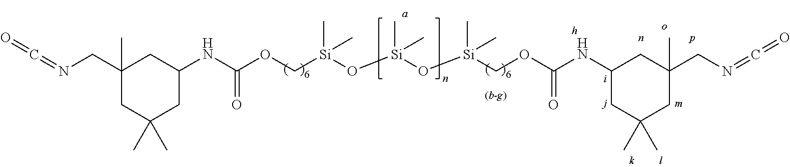

Isophorone diisocyanate (4.72 ml, 22.5 mmol) is introduced in a secured round-bottomed flask with reflux condenser and dropping funnel. Heating to 60° C. is performed under a protective gas atmosphere and with stirring. 0.1% by weight of triethylamine (32 mg) are added. Over the course of 2 h, α,ω-bis(hydroxyhexyl)poly(dimethylsiloxane) (n=30) (26.4 g, 11.0 mmol) is slowly added dropwise. Stirring is continued until there is no more clouding. The product is obtained in quantitative yield.

¹H-NMR (300 MHz, CDCl₃, 24° C.): δ [ppm]=3.98 (4H, m, H-g), 3.57 (2H, m, H-i), 2.98 (4H, m, H-p), 1.88-1.38 (8H, m, H-f/H-n), 1.26 (12H, m, H-c/H-d/H-e), 1.20-0.72 (26H, m, H-j/H-k/H-l/H-m/H-o), 0.46 (4H, m, H-b), 0.00 (168H, m, H-a); FT-IR (diamond): i [cm−1]=2961 (u R—CH3, SCH3, m-w), 2256 (u —NCO, isocyanate, s), 1709 (u C═O, urethane, s), 1412 (δ C—H, Si—CH3, w), 1257 (δ C—H, siloxane, s-m), 1011 (u Si—O—Si, siloxane, s-m); MALDI-TOF-MS m/z: 1542[M+Na]+ (for n=10), disubstituted, (¹H-NMR: with a to p as Ex. 4)

### Example 5

### Synthesis of Long-Chain α,ω-bis[hexyl(3-(isocyanatomethyl)-3,5,5-trimethylcyclohexyl)carbamyl]polydimethylsiloxane (PDMS-80-IPDI) (18), Structure Analogous to Example 4 where n=80

Isophorone diisocyanate (4.72 ml, 22.5 mmol) is introduced in a secured round-bottomed flask together with reflux condenser and dropping funnel. Heating to 60° C. is performed under a protective gas atmosphere and with stirring. 0.1% by weight of triethylamine (32 mg) are added. α,ω-bis(hydroxyhexyl)poly(dimethylsiloxane) (n=80) (67.1 g, 11.0 mmol) is slowly added dropwise over the course of 2 h. Stirring is continued until there is no more clouding. The product is obtained in quantitative yield.

¹H-NMR (300 MHz, CDCl₃, 24° C.): δ [ppm]=4.01 (4H, m, H-g), 3.77 (2H, m, H-i), 3.01 (4H, m, H-p), 1.89-1.42 (8H, m, H-f/H-n), 1.33 (12H, m, H-c/H-d/H-e), 1.24-0.76 (26H, m, H-j/H-k/H-l/H-m/H-o), 0.51 (4H, m, H-b), 0.00 (396H, m, H-a); FT-IR (diamond): ũ [cm−1]=2960 (u R—CH3, Si—CH3, m-w), 2256 (u —NCO, isocyanate, s), 1711 (u C═O, urethane, s), 1411 (δ C—H, Si—CH3, w), 1257 (δ C—H, siloxane, s-m), 1010 (u Si—O—Si, siloxane, s-m); MALDI-TOF-MS m/z: 2283[M+Na]+ (for n=20), disubstituted

### Example 6

### Synthesis of valine-terminated, short-chain polydimethylsiloxane (PDMS-30-Val) (20), n=30, FIG. 2

Valine methyl ester hydrochloride (0.75 g, 4.5 mmol) is dissolved in 20 ml of dichloromethane in a secured round-bottomed flask together with reflux condenser and dropping funnel. Triethylamine (0.625 ml, 4.5 mmol) is added. α,ω-bis[hexyl(3-(isocyanatomethyl)-3,5,5-trimethylcyclohexyl)carbamyl]poly(dimethylsiloxane) (n=30) (4.26 g, 1.5 mmol) is dissolved in 20 ml of dichloromethane and added dropwise by dropping funnel with stirring over the course of 1 h. After stirring for 24 h at RT, washing is performed with in each case 2×20 ml of dist. water and 1×20 ml of sat. aqueous sodium hydrogen carbonate solution. The organic phases are combined, dried over sodium sulfate and the solvent is removed on a rotary evaporator. The product is then dried in high vacuum.

¹H-NMR (600 MHz, CDCl₃, 24° C.): δ [ppm]=4.39 (2H, m, H-s), 4.01 (4H, m, H-g), 3.87 (2H, m, H-i), 3.71 (6H, m, H-w), 2.89 (4H, m, H-p), 2.10 (2H, m, H-t), 1.75-1.45 (8H, m, H-f/H-n), 1.31 (12H, m, H-c/H-d/H-e), 1.21-0.73 (38H, m, H-j/H-k/H-l/H-m/H-o/H-u/H-v), 0.46 (4H, m, H-b), 0.00 (174H, m, H-a); FT-IR (diamond): ũ [cm−1]=2961 (u R—CH3, Si—CH3, m-w), 2904 (u CH3, ester, w) (u C—H, free amino acid, m), 1726 (u C═O, ester, v) (u C═O, urethane, m-w), 1635 (u C═O, N—CO—N, v), 1561 (u C═O, N—CO—N, v), 1412 (5 C—H, Si—CH3, w), 1257 (5 C—H, siloxane, s-m), 1011 (u Si—O—Si, siloxane, s-m); MALDI-TOF-MS m/z: 1433[M+Na]+ (for n=5), disubstituted

### Example 7

### Synthesis of Valine-Terminated, Long-Chain Polydimethylsiloxane (PDMS-80-Val) (21), n=80, FIG. 2

Valine methyl ester hydrochloride (0.75 g, 4.5 mmol) is dissolved in 20 ml of dichloromethane in a secured round-bottomed flask together with reflux condenser and dropping funnel. Triethylamine (0.625 ml, 4.5 mmol) is added. α,ω-bis[hexyl(3-(isocyanatomethyl)-3,5,5-trimethylcyclohexyl)carbamyl]poly(dimethylsiloxane) (n=80) (9.81 g, 1.5 mmol) is likewise dissolved in 20 ml of dichloromethane and added dropwise per dropping funnel with stirring over the course of 1 h. After stirring for 24 h at RT, washing is performed with in each case 2×20 ml of dist. water and 1×20 ml of sat. aqueous sodium hydrogen carbonate solution. The organic phases are combined, dried over sodium sulfate and the solvent is removed on a rotary evaporator. The product is then dried in high vacuum.

¹H-NMR (600 MHz, CDCl₃, 24° C.): δ [ppm]=4.38 (2H, m, H-s), 4.01 (4H, m, H-g), 3.86 (2H, m, H-i), 3.71 (6H, m, H-w), 2.90 (4H, m, H-p), 2.10 (2H, m, H-t), 1.78-1.50 (8H, m, H-f/H-n), 1.31 (12H, m, H-c/H-d/H-e), 1.25-0.74 (38H, m, H-j/H-k/H-l/H-m/H-o/H-u/ H-v), 0.46 (4H, m, H-b), 0.00 (444H, m, H-a); FT-IR (diamond): ũ [cm−1]=2962 (u R—CH3, Si—CH3, m-w), 2907 (u CH3, ester, w) (u C—H, free amino acid, m), 1729 (u C═O, ester, v) (u C═O, urethane, m-w), 1641 (u C═O, N—CO—N, v), 1562 (u C═O, N—CO—N, v), 1412 (δ C—H, Si—CH3, w), 1257 (δ C—H, siloxane, s-m), 1009 (u Si—O—Si, siloxane, s-m); MALDI-TOF-MS m/z: 3507[M+Na]+ (for n=33), disubstituted, (¹H-NMR: with a to p as Ex. 4)

### Example 8

### Synthesis of Tryptophan-Terminated, Short-Chain Polydimethylsiloxane (PDMS-30-IPDI-Trp) (23), n=30

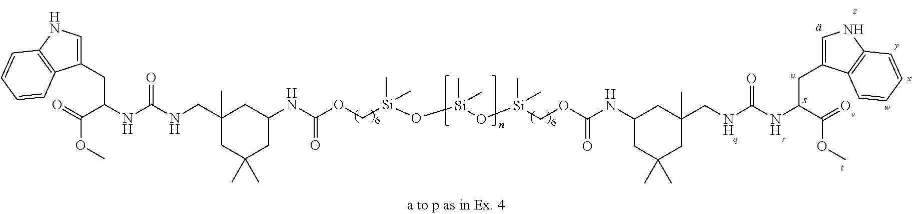

Tryptophan methyl ester hydrochloride (1.15 g, 4.5 mmol) is dissolved in a mixture of 20 ml of dichloromethane and 20 ml of dimethylformamide in a secured round-bottomed flask together with reflux condenser and dropping funnel. Triethylamine (0.625 ml, 4.5 mmol) is added. α,ω-bis[hexyl(3-(isocyanatomethyl)-3,5,5-trimethylcyclohexyl)carbamyl]poly(dimethylsiloxane) (n=30) (4.26 g, 1.5 mmol) is likewise dissolved in a mixture of 20 ml of dichloromethane and 20 ml of dimethylformamide and added dropwise per dropping funnel with stirring over the course of 1 h. After stirring for 24 h at RT, washing is performed with in each case 2×20 ml of dist. water and 1×20 mL of sat. aqueous sodium hydrogen carbonate solution. The organic phases are combined, dried over sodium sulfate and the solvent is removed on a rotary evaporator. The crude product is dissolved in some ethanol, precipitated again in dist. water and centrifuged. Finally, the product is dried in high vacuum.

¹H-NMR (600 MHz, CDCl₃, 24° C.): δ [ppm]=7.46 (2H, m, H-v), 7.28 (4H, m, H-y), 7.04 (6H, m, H-w/H-x/H-a), 4.72 (2H, m, H-s), 3.96 (4H, m, H-g), 3.63 (12H, m, H-i/H-t/H-u), 3.20 (4H, m, H-p), 1.67-1.39 (8H, m, H-f/H-n), 1.26 (12H, m, H-c/H-d/H-e), 1.17-0.59 (26H, m, H-j/H-k/H-l/H-m/H-o), 0.46 (4H, m, H-b), 0.00 (144H, m, H-a) FT-IR (diamond): ũ [cm−1]=2961 (u R—CH3, Si—CH3, m-w), 2600 (u C—H, free amino acid, m), 2495 (u C—H, free amino acid, m), 1667 (u C═O, N—CO—N, v) (u C═O, urethane, m-w), 1439 (δ C—H, Si—CH3, w), 1258 (δ C—H, siloxane, s-m), 1015 (u Si—O—Si, siloxane, s-m); MALDI-TOF-MS m/z: 2126[M+Na]+ (for n=12), disubstituted

### Example 9

### Synthesis of Tryptophan-Terminated, Long-Chain Polydimethylsiloxane (PDMS-80-IPDI-Trp) (24), Structure Analogous to Example 8, n=80

Tryptophan methyl ester hydrochloride (1.15 g, 4.5 mmol) is dissolved in a mixture of 20 ml of dichloromethane and 20 ml of dimethylformamide in a secured round-bottomed flask with reflux condenser and dropping funnel. Triethylamine (0.625 ml, 4.5 mmol) is added. α,ω-bis[hexyl(3-(isocyanatomethyl)-3,5,5-trimethylcyclohexyl)carbamyl]-poly(dimethylsiloxane) (n=80) (9.81 g, 1.5 mmol) is dissolved in a mixture of 20 ml of dichloromethane and 20 ml of dimethylformamide and added dropwise with stirring over the course of 1 h. After stirring for 24 h at RT, washing is performed with in each case 2× 20 ml of dist. water and 1×20 ml of sat. aqueous sodium hydrogen carbonate solution. The organic phases are combined, dried over sodium sulfate and the solvent is removed on a rotary evaporator. The crude product is dissolved in some ethanol, precipitated again in dist. water and centrifuged. The product is dried in high vacuum.

¹H-NMR (600 MHz, CDCl₃, 24° C.): δ [ppm]=7.51 (2H, m, H-v), 7.33 (4H, m, H-y), 7.05 (6H, m, H-w/H-x/H-ä), 4.78 (2H, m, H-s), 4.01 (4H, m, H-g), 3.68 (12H, m, H-i/H-t/H-u), 3.24 (4H, m, H-p), 1.81-1.41 (8H, m, H-f/H-n), 1.31 (12H, m, H-c/H-d/H-e), 1.16-0.62 (26H, m, H-j/H-k/H-l/H-m/H-o), 0.51 (4H, m, H-b), 0.00 (366H, m, H-a); FT-IR (diamond): ü [cm−1]=2959 (u R—CH3, Si—CH3, m-w), 2600 (u C—H, free amino acid, m), 2495 (u C—H, free amino acid, m), 1666 (u C═O, N—CO—N, v) (u C═O, urethane, m-w), 1439 (δ C—H, Si—CH3, w), 1258 (δ C—H, siloxane, s-m), 1087 (u Si—O—Si, siloxane, s-m); MALDI-TOF-MS m/z: 3165[M+Na]+ (for n=26), disubstituted

### Example 10

### Synthesis of Histidine-Terminated, Long-Chain Polydimethylsiloxane (PDMS-80-IPDI-His)

The preparation takes place according to the experimental procedure in example 9 Using histidine methyl ester hydrochloride (1.15 g, 4.5 mmol).

### Example 11

### Synthesis of Histidine-Terminated, Short-Chain Polydimethylsiloxane (PDMS-30-IPDI-His)

The preparation takes place according to the experimental procedure of example 8 using histidine methyl ester hydrochloride (1.15 g, 4.5 mmol).

### Example 12

### Synthesis of α,ω-bis[hexyl(6-isocyanatohexyl)carbamyl]poly(dimethylsiloxane)

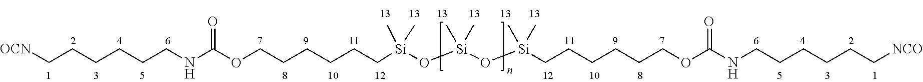

2.00 g (11.89 mmol) of 1,6-hexamethylene diisocyanate, dissolved in 20 ml of ethyl acetate, are introduced in a baked-out, argon-flushed and secured apparatus with reflux condenser and dropping funnel. Heating is carried out to 75° C. with stirring and then 0.1% by weight (16 mg) of triethylamine are added. A dropping funnel is then used to slowly add 14 g (5.8 mmol) of α,ω-bis(hydroxyhexyl)poly(dimethylsiloxane), dissolved in 30 ml of ethyl acetate, to the diisocyanate component (dropwise addition time 2 h). During this, stirring is continuous. Stirring is then performed for a further 16 h at 75° C. Yield: 15.90 g

¹H-NMR: (300 MHz, CDCl₃) δ=4.01 (m, 4H, 7), 3.27 (q, 4H, 1), 3.13 (m, 4H, 6), 1.68-1.23 (m, 32H, 2, 3, 4, 5, 8, 9, 10, 11), 0.50 (m, 4H, 12), 0.04 (m, 180H, 13) ppm. FT-IR (diamond): \tilde{v}=2964 v(C—H), 2267 v(NCO), 1708 v(C═O)_{urethane,} 1523 δ(N—H), 1410 δₐₛ(C—H), 1254 δ_{sym}(C—H), 1011 v(Si—O—C), 853+789 v(SI—C) cm⁻¹.

### Example 13

### Synthesis of α,ω-bis[hexyl(6-(valine methyl ester)ureylhexyl)carbamyl]-poly(dimethylsiloxane)

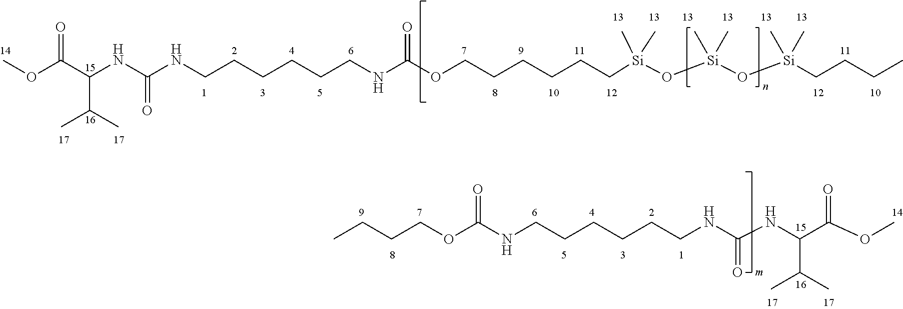

168 mg (1 mmol) of valine methyl ester hydrochloride are dissolved in 15 ml of dichloromethane and introduced in a baked-out, argon-flushed and secured apparatus with reflux condenser and dropping funnel. 101.2 mg

(0.139 ml, 1 mmol) of triethylamine are added. 1.033 g (0.378 mmol) of α,ω-bis[hexyl(6-isocyanatohexyl)carbamyl]poly(dimethylsiloxane) are dissolved in 20 ml of dichloromethane and added to the amino acid per dropping funnel with stirring over the course of one hour. After stirring for two hours at room temperature, the organic phase is washed 3× with in each case 15 ml dH₂O, 1× with 15 ml sat. aqueous NaHCO₃ solution and 1× with 15 ml sat. NaCl solution. The organic phases are combined, dried over Na₂SO₄ and the solvent is removed on a rotary evaporator. Drying is then carried out in a high vacuum. Yield: 0.92 g (79%)

¹H-NMR: (300 MHz, CDCl₃) δ=4.36 (m, 0.85H, 15), 4.01 (m, 4H, 7), 3.71 (m, 2.71H, 14) 3.31-3.30 (m, 8H, 1, 6), 2.10 (m, 0.89H, 16), 1.68-1.23 (m, 32H, 2, 3, 4, 5, 8, 9, 10, 11), 0.98-0.82 (m, 5.52H, 17), 0.50 (m, 4H, 12), 0.04 (m, 180H, 13) ppm.

FT-IR (diamond): \tilde{v}=2962 v(C—H), 1720 v(C═O)ᵤᵣₑₜₕₐₙₑ, 1658 v(C═O)ᵤᵣₑₐ, 1563 δ(N—H), 1404 δₐₛ(C—H), 1257 δ_{sym}(C—H), 1011 v(Si—O—C), 863+789 v(SI—C) cm⁻¹.

### Example 14

### Synthesis of Isocyanate-Functionalized Amino Acid Derivatives (A-IPDI, IXa*, IXb*)

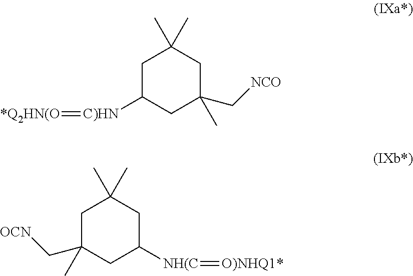

and optionally of the general formulae IXc* and/or IXd* or mixtures thereof.

### Valinemethyl Ester-IPDI

Isophorone diisocyanate (4.72 mL, 22.5 mmol) is introduced in a previously secured apparatus at RT. 1-2 drops of triethylamine are added under an argon countercurrent. A dropping funnel is used to add valine methyl ester hydrochloride (22.5 mmol), dissolved in 10 ml of dichloromethane and 10 ml of dimethylformamide, dropwise to the reaction mixture over the course of 2 h. After stirring for 24 h at RT, the solvents are removed on a rotary evaporator and the product is finally dried in a high vacuum. The valine methyl ester IPDI is obtained.

### Example 16

### Synthesis of an Isocyanate-Functionalized Amino Acid Derivative (A-IPDI), Histidine Methyl Ester IPDI

Isophorone diisocyanate (4.72 mL, 22.5 mmol) is introduced in a previously secured apparatus at RT. 1-2 drops of triethylamine are added under an argon countercurrent. A dropping funnel is used to add histidine methyl ester hydrochloride (22.5 mmol), dissolved in 10 ml of dichloromethane and 10 ml of dimethylformamide, dropwise to the reaction mixture over the course of 2 h. After stirring for 24 h at RT, the solvents are removed on a rotary evaporator and the product is finally dried in a high vacuum. The histidine methyl ester IPDI is obtained.

### Application Test—Combing Force

For the applications-related assessment of the conditioning of hair, the compounds according to the invention specified below, as well as the commercially available product ABIL® Quat 3272, were used in a simple cosmetic hair rinse formulation and investigated by means of combing force measurements.

The formulation constituents are named in the compositions in the form of the generally recognized INCI nomenclature using the English terms. All concentrations are given in the application examples in percent by weight.

For the applications-related assessment, hair tresses (brown Caucasian flat tresses, untreated, Kerling Germany) were predamaged in a standardized way by means of a perming treatment (“universal perm” with “foam neutralizer concentrate”, Basler). 4 g of perming composition/g of hair, contact time 15 min, rinsing time 2 min under running water (T=38° C.). Then 4 g of neutralizer solution (1 part foam neutralizer+3 parts water)/g of hair, contact time 10 min, rinsing time 2 min under running water. For this, customary hairstyling products were used.

The combing force measurement was carried out using the diastron MTT 175 instrument (speed: 2000 mm/min) under standardized conditions (22° C., 50% relative humidity).

The hair was pretreated using a shampoo which contained no conditioners.

Standardized treatment and measurement of the hair tresses: The hair tresses were stored under controlled conditions (22° C., 50% relative humidity) for at least 12 h. The hair tresses were then dipped for 1 min in a highly diluted buffer (Na-citrate, pH-6) solution. The hair was then precombed by hand until no detectable improvement in combability can be achieved. The hair is hung up until the residual moisture in the hair was approx. 60% (+−5%) (2 g hair+1.2 g residual moisture). The hair tress was then fixed on the measuring instrument and the first combing force measurement was started. The measurement was repeated 10 times. Before each measurement, the hair tress was wetted by spraying twice with the citrate buffer solution using a spray bottle.

The average value of the combing force measurements was determined via the MTT175 software. For statistical reasons, 4 hair tresses were measured for each formulation. The same procedure and measurement was carried out before and after treating the hair with the hair rinse.

For the standardized treatment of the hair tresses with the hair rinse, the predamaged hair tresses were treated as follows with the conditioning rinse described below:

The hair tresses were wetted under running, 38° C. hot water. The excess water was gently squeezed out by hand, then the rinse was applied and gently worked into the hair for 1 min (0.5 g/hair tress (2 g)). After a contact time of 5 min, the hair was rinsed for 1 min.

For the evaluation of the measurements, the difference in the required combing forces before treatment compared to after treatment with the hair rinse was calculated.

The siloxanes according to the invention are applied to the hair as explained above in a hair rinse of the formulation given below, and rinsed out of the treated hair for one minute. The reduction in the force to be applied for combing a defined hair sample is then measured. The siloxanes according to the invention reduce the force by about 51 to 56%, with the siloxane where n=80, IPDI+methyltryptophan showing the best results. The matrix formulation (CTAC) comprises 0.5% by weight of ceteareth-25, 5.0% by weight of cetyl alcohol, 1.0% by weight of cetrimonium chloride, ad 100% water, the pH is about 4.3. The formulations according to the invention moreover comprise, in 100% by weight, 0.5% by weight of the siloxanes functionalized according to the invention with amino acids, the pH is about 4.3. Similarly, the siloxane where n=80+IPDI+histidine, and the siloxane where n=30, +IPDI+methyltryptophan exhibit a reduction in the combing force by about 51%. The pH can be regulated with citric acid. As comparison, 1.0% by weight of ABIL® Quat 3272 (siliconequat from Evonik Industries, 50% strength in propylene glycol) was used in the matrix formulation.

Table 1 below shows the results of the combing force measurements as reduction in the combing force to be applied for the above matrix formulation with in each case 0.5% by weight of the functionalized PDMS according to the invention of the general formula I.

TABLE 1
Combing force reduction

| | Combing force reduction |
|---|---|
| | (comparison of before and after |
| | a treatment with the hair rinse) |
| CTAC (matrix formulation) | 35.3% |
| CTAC + ABIL QUAT 3272 | 49% |
| CTAC + (PDMS-80-Val) (21) | 51.3% |
| CTAC + PDMS-80-IPDI-His) | 54.1% |
| CTAC + (PDMS-30-IPDI-Trp) (23) | 51.7% |
| CTAC + PDMS-80-IPDI-Trp) (24) | 56.7% |

While this invention has been described in conjunction with the specific embodiments outlined above, it is evident that many alternatives, modifications, and variations will be apparent to those skilled in the art. Accordingly, the preferred embodiments of the invention as set forth above are intended to be illustrative, not limiting. Various changes may be made without departing from the spirit and scope of the inventions as defined in the following claims.

1. A siloxane polymer of the general formula I comprising a central polysiloxane polymer block B,
(i) which is substituted with organofunctional radicals,
(ii) the polymer block B has linear and/or branched structures with at least two difunctional siloxane units,
(iii) the polymer block B has on at least two terminal silicon atoms or at least one terminal and at least one lateral silicon atom on the siloxane units of polymer block B, the organofunctional radicals -Q1 and -Q2, where the radicals are identical or different,
Q2-B-Q1 (I)
where -Q1 corresponds to the general formula IIa and -Q2 corresponds to the formula IIb,
-Q1=-Q1′-A-(C═O)-D-Q1″-A′-(C═O)-D′-Q1* (IIa)
-Q2=-Q2′-A-(C═O)-D-Q2″-A′-(C═O)-D′-Q2* (IIb)
where A is —NH—, —O— or —S— and D is —NH—, in each case independently in formulae IIa and IIb,
where A′ is —NH— and D′ is —NH—, —O— or —S—, in each case independently in formulae IIa and IIb, where each radical Q1 and Q2 of the formulae IIa or IIb has at least one bivalent urea group and one further bivalent urea or carbamate group
with Q1′ and Q2′ in each case independently comprising a bivalent hydrocarbon radical with 6 to 200 carbon atoms optionally comprising at least one heteroatom selected from O, N and S, a bivalent radical comprising aryl, arylalkyl groups optionally comprising at least one heteroatom O, N or S or polyether radicals containing alkyl, aryl or alkyl and aryl groups,
with Q1″ and Q2″ in each case independently comprising a bivalent linear, branched and/or cyclic alkyl radical having 4 to 200 carbon atoms or a bivalent radical comprising an aryl and/or arylalkyl radical with 6 to 200 carbon atoms, and
with -D′-Q1* and -D′-Q2* with D′, as defined above, and where -D′-Q1* and -D′-Q2* in each case independently comprise radicals which are derived from an amino acid, an amino acid derivative or salts thereof.

2. The siloxane polymer according to claim 1, wherein
in the siloxane polymer of the general formulae I, IIa and IIb -D′-Q1* and -D′-Q2* in each case independently form radicals which are derived from an amino acid, an amino acid derivative or salts thereof and D′ is —NH in each case independently in -D′-Q1* and -D′-Q2* is in the alpha position relative to an ester or a carboxyl group of the amino acid, of the amino acid derivative or salt, where the ester includes alkyl esters with 1 to 25 carbon atoms or aryl esters.

3. The siloxane polymer according to claim 1, wherein
in the siloxane polymer of the general formula I, the polymer block B corresponds to the general formula IIIa or IIIb, where B is 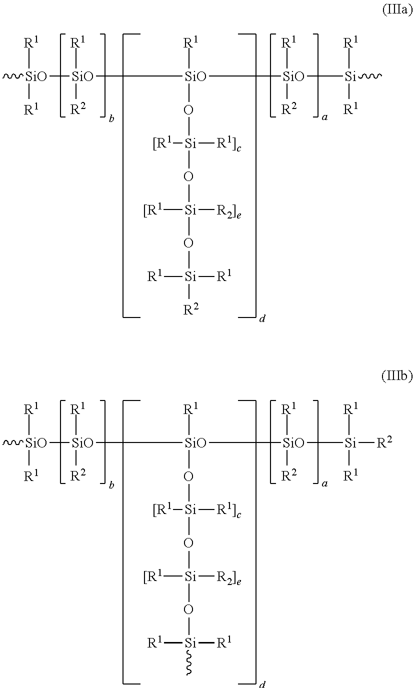
where a, b, c, d and e in formulae IIIa and IIIb are in each case independently an integer
where a is from 1 to 200,
where b is from 0 to 200,
where c is from 0 to 200,
where d is from 0 to 200,
where e is from 0 to 200 and
where R¹ in formulae IIIa or IIIb are in each case independently identical or different, where R¹ comprises alkyl radicals with 1 to 22 carbon atoms, or phenyl radicals,
where R² in formulae IIIa or IIIb is an alkyl radical with 1 to 22 carbon atoms, an alkyl radical with at least one heteroatom selected from N, O, S or a phenyl radical.

4. The siloxane polymer according to claim 1, wherein
b, c, d and e are 0 and a is 20 to 100.

5. The siloxane polymer according to claim 1, wherein
R¹ and R² are selected from alkyl groups with 1, 2, 3 or 4 carbon atoms.

6. The siloxane polymer according to claim 1, wherein
the radicals -Q1 and -Q2 in the general formula I are independently selected from
-Q1=-Q1′-A-(C═O)-D-Q1″-A′-(C═O)-D′-Q1* (IIa)
-Q2=-Q2′-A-(C═O)-D-Q2″-A′-(C═O)-D′-Q2* (IIb)
a) where A is —O—, D is —NH—, A′ is —NH— and D′ is —NH—,
b) where A is —NH—, D is —NH—, A′ is —NH— and D′ is —NH—,
c) where A is —, D is —NH—, where A′ is —NH— and D′ is —NH—,
d) where A is —O—, D is —NH—, A′ is —NH— and D′ is —O—,
e) where A is —NH—, D is —NH—, A′ is —NH— and D′ is —O—,
f) where A is —S—, D is —NH—, A′ is —NH— and D′ is —O—,
g) where A is —O—, D is —NH—, A′ is —NH— and D′ is —S—,
h) where A is —NH—, D is —NH—, A′ is —NH— and D′ is —S— or
i) where A is —S—, D is —NH—, A′ is —NH— and D′ is —S—.

7. The siloxane polymer according to claim 1, wherein
in the radicals -Q1 and -Q2 of the formula I, the radicals -D′-Q1* and -D′-Q2* are in each case independently derived from amino acids, amino acid derivatives or salts thereof, comprising
nonpolar amino acids comprising alanine, valine, methionine, leucine, isoleucine, proline, tryptophan, phenylalanine,
basic amino acids comprising lysine, arginine, histidine,
polar and neutral amino acids tyrosine, threonine, glutamine, glycine, serine, cysteine, asparagine and/or
acidic amino acids selected from glutamic acid and aspartic acid,
and their mono-, dicarboxylic acid esters, amides with primary amino groups of the amino acids, amides of the carboxylic acid groups of the amino acids and/or esters with the primary hydroxyl groups, thioesters of the HS groups or amides with secondary amino groups of the amino acids.

8. The siloxane polymer according to claim 1, wherein
in the radicals -Q1 and -Q2 of the formula I,
A is —O—, D is —NH—, A′ is —NH— and D′ is —NH—, —O— or —S— and the radicals -D′-Q1* and -D′-Q2* are in each case independently derived from amino acids, amino acid derivatives or salts thereof, amides with primary amino groups of the amino acids, amides of the carboxylic acid groups of the amino acids and/or esters with the primary hydroxyl groups or thioesters of the HS group, and salts thereof.

9. The siloxane polymer according to claim 1, wherein
in the radicals -Q1 and -Q2 of the formula I, the bivalent radicals -Q1″- and -Q2″- are independently selected from bivalent, linear, branched or cyclic alkylene radicals with 4 to 25 carbon atoms.

10. The siloxane polymer according to claim 1, wherein
in the radicals -Q1 and -Q2 of the formula I, the bivalent radicals -Q1′- and -Q2′- are selected from alkylene radicals with 3 to 22 carbon atoms optionally with at least one heteroatom comprising N, O or S, or from polyether radicals containing alkyl, aryl or alkyl and aryl groups of the formulae IVa or IVb
where Q1′ and Q2′ are in each case independently
-T-O—(CH₂—CH₂—O—)ₓ—(CH₂—CH(R#)O—)_{y}(SO)—R″ (IVa)
-T-O—(CH₂—CH₂—O—)ₓ—(CH₂—CH(R#)O—)—R″ (IVb),
where T=bivalent hydrocarbon radical with 2 to 4 carbon atoms,
where x=0 to 200, y=0 to 200, where x and y are integers, with the proviso that x or y is at least 1,
where R# is hydrogen or methyl, and
R″ is linear or branched alkylene.

11. The siloxane polymer according to claim 1, wherein
in the radicals -Q1 and -Q2 of the formula (I), at least one of the bivalent radicals -Q1″- and -Q2″- is independently a bivalent cyclohexane-containing radical selected from the formulae Va and Vb (Va) (Vb)

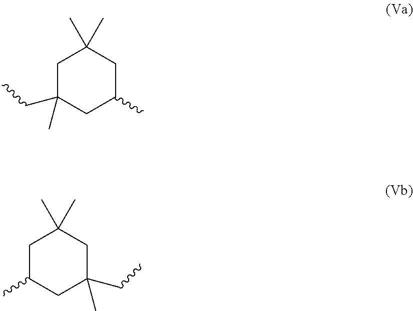

12. The siloxane polymer according to claim 1, wherein
the siloxane of the general formula I corresponds to the siloxane polymer of the general formula XI where
Mₐ₁M^{A}ₐ₂M^{B}ₐ₃D_{b1}D^{A}_{b2}D^{B}_{b3}T_{c1}T^{A}_{c}2T^{B}_{c3}Q_{d1} (XI)
where M=[R¹⁶₃SiO_{1/2}], M^{A}=[R¹⁷R¹⁶₂SiO_{1/2}], M^{B}=[R¹⁸R¹⁶₂SiO_{1/2}],
where D=[R¹⁶₂SiO_{2/2}], D^{A}=[R¹⁷₁R¹⁶₁SiO_{2/2}], D^{B}=[R¹⁸1R¹⁶1SiO_{2/2}],
where T=[R¹⁶SiO_{3/2}], T^{A}=[R¹⁷SiO_{3/2}], T^{B}=[R¹⁸SiO_{3/2}], Q=[SiO_{4/2}],
where R¹⁶, independently of one another, are identical or different linear or branched, saturated or unsaturated hydrocarbon radicals having 1 to 30 carbon atoms or else aromatic hydrocarbon radicals having 6 to 30 carbon atoms, where R¹⁷ is in each case independently -Q1 or -Q2,
where R¹⁸ independently of one another are identical or different linear or branched, saturated or olefinically unsaturated hydrocarbon radicals with 8 to 30 carbon atoms, aromatic hydrocarbon radical with 6 to 40 carbon atoms, alkylaryl radical with 7 to 40 carbon atoms, a linear or branched optionally double-bond-containing aliphatic hydrocarbon radical with 2 to 30 carbon atoms that is interrupted by one or more heteroatoms, such as oxygen, NH, NR′ where R′ is an optionally double-bond-containing C1 to C30-alkyl radical, a linear or branched optionally double-bond-containing aliphatic hydrocarbon radical with 2 to 30 carbon atoms interrupted by one or more functionalities selected from the group —OH, —O—C(O)—, —(O)C—O—, —NH—C(O)—, —(O)C—NH, —(CH₃)N—C(O)—, —(O)C—N(CH₃)—, —S(O₂)—O—, —O—S(O₂)—, —S(O₂)—NH—,
—NH—S(O₂)—, —S(O₂)—N(CH₃)—, —N(CH₃)—S(O₂)—, a linear or branched optionally double-bond-containing aliphatic or cycloaliphatic hydrocarbon radical with 1 to 30 carbon atoms terminally functionalized with OH, OR′, NH², N(H)R′, N(R′)₂ where R′ is an optionally double-bond-containing C1 to C30-alkyl radical, or a blockwise or randomly constructed polyether according to (R⁵—O)ₙ—R⁶, where R⁵ is a linear or branched hydrocarbon radical containing 2 to 4 carbon atoms, n is 1 to 100, and R⁶ is hydrogen, a linear or branched optionally double-bond-containing aliphatic hydrocarbon radical with 1 to 30 carbon atoms, an optionally double-bond-containing cycloaliphatic hydrocarbon radical with 5 to 40 carbon atoms, an aromatic hydrocarbon radical with 6 to 40 carbon atoms, an alkylaryl radical with 7 to 40 carbon atoms, or a radical —C(O)—R⁷ where R⁷ is a linear or branched optionally double-bond-containing aliphatic hydrocarbon radical with 1 to 30 carbon atoms, an optionally double-bond-containing cycloaliphatic hydrocarbon radical with 5 to 40 carbon atoms, an aromatic hydrocarbon radical with 6 to 40 carbon atoms, an alkylaryl radical with 7 to 40 carbon atoms with the indices
a1=0-200,
a2=0-30,
a3=0-30, b1=2 to 5000, b2=0 to 100,
b3=0 to 100, c1=0 to 30,
c2=0 to 30,
c3=0 to 30,
d1=0 to 30,
with the proviso that at least one of the indices selected from a2 and a3 is not equal to 0.

13. The siloxane polymer according to claim 1, wherein
the siloxane polymer is selected from siloxane polymers of the formulae Ia and Ib or mixtures of these 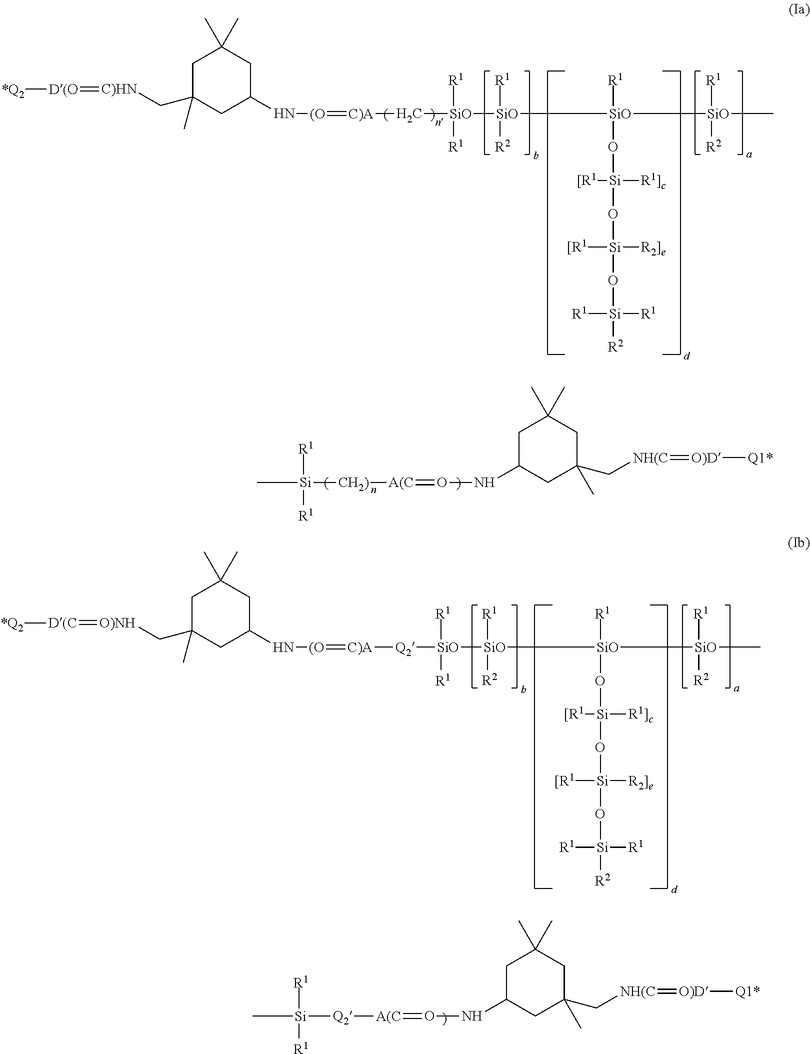
where n or n′ is in each case independently selected from an integer from 3 to 22,
where a is from 1 to 200,
where b is from 0 to 200,
where c is from 0 to 200,
where d is from 0 to 200,
where e is from 0 to 200 and
where R′ in formulae Ia and Ib are in each case independently identical or different,
where R¹ comprises alkyl radicals with 1 to 4 carbon atoms or phenyl radicals,
where R² is alkyl radical with 1 to 22 carbon atoms, an alkyl radical with at least one heteroatom selected from N, O, S or phenyl radical and
where -D′-Q1* and -D′-Q2* are in each case independently derived from amino acids, amino acid derivatives or salts thereof, and
where Q1′ and Q2′ in formula Ib are in each case independently alkylene with 2 to 40 carbon atoms or a polyether of the formulae IVa or IVb
-T-O—(CH₂—CH₂—O—)—(CH₂—CH(R)O—)—(SO)—R″ (IVa)
-T-O—(CH₂—CH₂—O—)—(CH₂—CH(R)O—)—R″ (IVb)
where T=bivalent hydrocarbon radical with 2 to 4 carbon atoms,
where x=0 to 200 and y=0 to 200, where x and y are integers, with the proviso that x or y is at least 1,
where R# is hydrogen or methyl, and
where R″ is hydrogen or linear or branched alkylene.

14. The siloxane polymer according to claim 1, wherein
the siloxane polymer is selected from siloxane polymers of the formula Ia* and Ib* 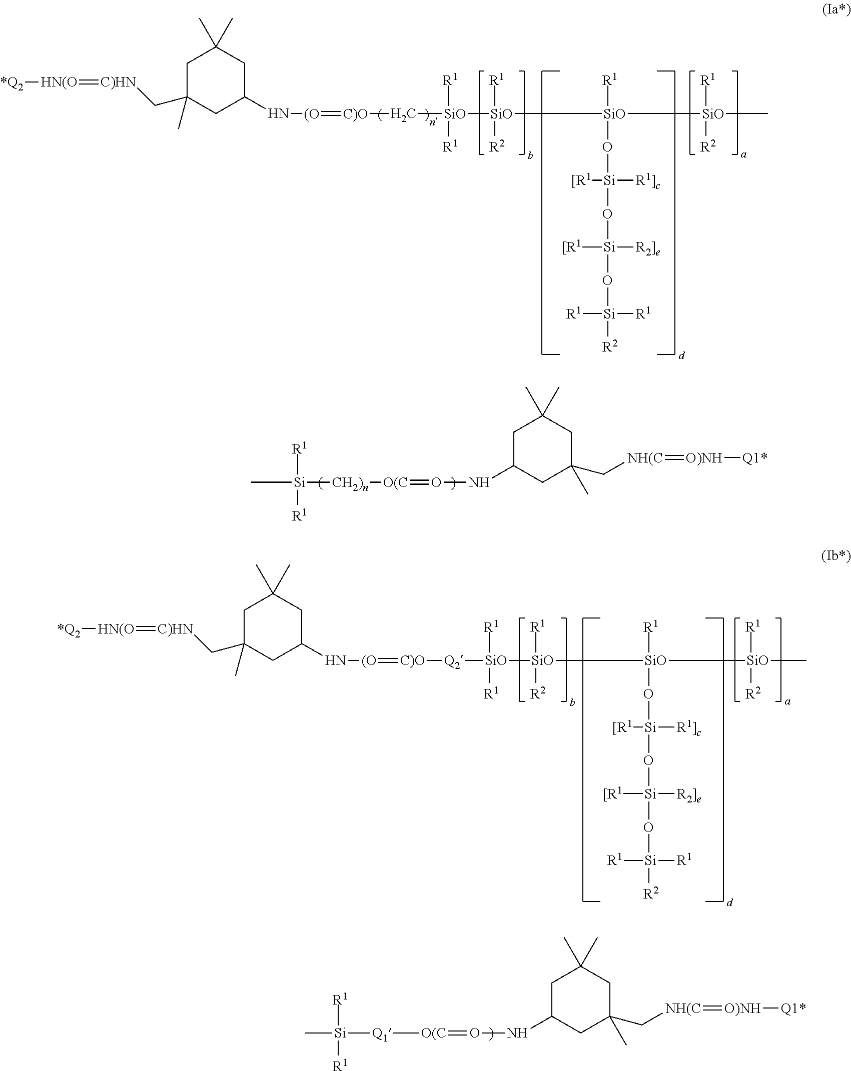
where n or n′ in each case independently is selected from an integer from 3 to 22,
where a is from 1 to 200,
where b is from 0 to 200,
where c is from 0 to 200,
where d is from 0 to 200,
where e is from 0 to 200 and
where R′ in formulae Ia* and Ib* is in each case independently identical or different, where R¹ comprises alkyl radicals with 1 to 4 carbon atoms or phenyl radicals,
where R² is alkyl radical with 1 to 22 carbon atoms,
an alkyl radical with at least one heteroatom comprising N, O, S or phenyl radical and where Q1* and Q2* are in each case independently selected from amino acids, amino acid derivatives or salts thereof, where the fragment —NH-Q1* and —NH-Q2*, as a result of reaction of the secondary alpha-amino groups of the aminoacids, derivatives thereof or the salts with the isocyanate, forms a urea group, and where the amino acids comprising nonpolar amino acids comprising alanine, valine, methionine, leucine, isoleucine, proline, tryptophan, phenylalanine, basic amino acids comprising lysine, arginine, histidine, polar and neutral amino acids comprising tyrosine, threonine, glutamine, glycine, serine, cysteine, asparagine and/or the acidic amino acids selected from glutamic acid and aspartic acid, and their mono-, dicarboxylic acid esters, amides with primary amino groups of the amino acids, amides of the carboxylic acid groups of the amino acids and/or esters with the primary hydroxyl groups or thioesters of the HS group or salts thereof, and in formula Ib* where Q1′ and Q2′ are in each case independently
-T-O—(CH₂—CH₂—O—)—(CH₂—CH(R#)O—)_{y}(SO)—R″ (IVa)
-T-O—(CH₂—CH₂—O—)—(CH₂—CH(R#)O—)_{y}—R″ (IVb),
where T=bivalent hydrocarbon radical with 2 to 4 carbon atoms,
where x=0 to 200, y=0 to 200, where x and y are integers with the proviso that x or y is at least 1,
where R# is hydrogen or methyl, and
where R″ is hydrogen or linear or branched alkylene.

15. A process for the preparation of a siloxane polymer and of compositions comprising these siloxane polymers or mixtures of the siloxane polymers with a central polysiloxane polymer block B, by reacting
a) a polysiloxane diisocyanate of the formula VII, with at least one amino acid, an amino acid derivative, salt thereof or mixtures thereof, giving a siloxane polymer of the general formula I
OCN-Q2″-D-(O═C)-A-Q2′-B-Q1′-A-(C═O)-D-Q1″-NCO (VII)
Q2-B-Q1 (I)
where -Q1 corresponds to the general formula IIa and -Q2 corresponds to the formula IIb,
-Q1=-Q1′-A-(C═O)-D-Q1″-A′-(C═O)-D′-Q1* (IIa)
-Q2=-Q2′-A-(C═O)-D-Q2″-A′-(C═O)-D′-Q2* (IIb)
where A is —NH—, —O— or —S— and D is —NH— in each case independently in formulae IIa and IIb,
where A′ is —NH— and D′ is —NH—, —O— or —S— in each case independently in formulae IIa and IIb, where each radical Q1 and Q2 of the formula IIa or IIb in each case independently has at least one bivalent urea group and one further bivalent urea or a carbamate group, or
b) a polysiloxane of the formula VI, with an amino acid isocyanate selected from the formulae IXa, IXb, IXc, IXd or mixtures thereof
HA-Q2′-B-Q1′-AH (VI)
Q2*—NH(CO)NH-″2Q-NCO (IXa)
Q1*—NH(CO)NH-″1-NCO (IXb)
Q2*—NH(CO)NH-Q2″-NCO (IXc)
Q1*—NH(CO)NH-Q1″-NCO (IXd)
where A is —NH—, —O— or —S— and D is —NH— in each case independently in formulae VII, I and VI,
where Q1′ and Q2′ in each case independently comprising a bivalent hydrocarbon radical with 6 to 200 carbon atoms optionally comprising at least one heteroatom O, N or S, a bivalent radical comprising aryl, arylalkylgroups or a bivalent radical comprising aryl, arylalkyl groups optionally comprising at least one heteroatom O, N or S or polyether radicals containing alkyl, aryl or alkyl and aryl groups, in each case independently in formulae VII, I and VI,
where Q1″ and Q2″ in each case independently comprises a bivalent linear, branched and/or cyclic alkyl radical with 4 to 200 carbon atoms, or a bivalent radical comprising an aryl and/or arylalkyl radical with 6 to 200 carbon atoms, in each case independently in formulae VII, I, IXa, IXb, IXc and IXd
where —NH-Q1* and —NH-Q2* comprises in each case independent radicals which are derived from an amino acid, amino acid derivative or salt thereof.

16. The process according to claim 15, wherein
formulae IXa, IXb, IXc and/or IXd and I—NH-Q1* and —NH-Q2* in each case independently comprise radicals which are derived from an amino acid, amino acid derivative or salt thereof and —NH in —NH-Q1* and —NH-Q2* in each case is in the alpha position relative to an ester or a carboxyl group of the amino acid, the amino acid derivative or salt thereof, where the ester comprises alkyl ester with 1 to 25 carbon atoms or aryl ester.

17. The process according to claim 15, wherein
the polysiloxane of the formula VI is reacted with a diisocyanate to give a polysiloxane diisocyanate of the formula VII,
HA-Q2′-B-Q1′-AH (VI)
OCN-Q2″-D-(O═C)-A-Q2′-B-Q1′-A-(C═O)-D-Q1″-NCO (VII)
where A is selected from —O, —NH, —S—
where -AH is selected from —OH, —NH₂, and —SH and
where -Q2″- and/or -Q1″- independently selected from a bivalent, linear, branched and/or cyclic alkyl radical with 4 to 200 carbon atoms, or a bivalent radical comprising an aryl and/or arylalkyl radical with 6 to 200 carbon atoms, where the molar ratio of HA groups in the polysiloxane to isocyanate groups in formula VII is at least 1:1.

18. The process according to claim 15, which comprises
reacting
(i) a) a polysiloxane-group-containing linear and/or branched polymer block B, with at least two terminal Si—H groups or at least one terminal Si—H group and at least one lateral Si—H group, or b) a polysiloxane group of the formula XI with at least one
R¹⁷ is hydrogen,
with (ii) an olefinic compound comprising alkylene and optionally comprising at least one heteroatom N and/or O,
where the olefinic compound has in each case independently an allyl or vinyl group and corresponds to the formulae VIIIa and/or VIIIb
Q1′-AH (VIIIa)
Q2′-AH (VIIIb)
in the presence of
(iii) a catalyst to give a polysiloxane of the formula VI
HA-Q2′-B-Q1′-AH (VI)
where in each case independently in formulae VIIIa, VIIIb and VI with AH independently selected from —OH and —NH₂ and with -Q2′- and -Q1′- in each case independently in formulae VIIIa, VIIIb and VI comprising a bivalent hydrocarbon radical with 6 to 200 carbon atoms optionally comprising at least one heteroatom O or N a bivalent radical comprising aryl, arylalkyl groups optionally comprising at least one heteroatom O or N olefinic polyether.

19. The process according to claim 15, wherein
a polysiloxane diisocyanate of the formula VII is reacted with an amino acid, amino acid derivative, salts thereof or mixtures thereof,
OCN-Q2″-D-(O═C)-A-Q2′-B-Q1′-A-(C═O)-D-Q1″-NCO (VII)
where B is a linear and/or branched polysiloxane polymer block B, with -Q2′- and -Q1′- in each case independently comprising a bivalent hydrocarbon radical with 6 to 200 carbon atoms optionally comprising at least one heteroatom O, N, or S, a bivalent radical comprising aryl, arylalkyl groups optionally comprising at least one heteroatom O, N or S, or polyether radicals containing alkyl, aryl or alkyl and aryl groups, where A is in each case independently —NH—, —O— or —S— and D is —NH— in each case independently in formula VII, and where -Q2″- and/or -Q1″- are independently selected from a bivalent, linear, branched and/or cyclic alkyl radical with 4 to 200 carbon atoms, or a bivalent radical comprising an aryl and/or arylalkyl radical with 6 to 200 carbon atoms,

20. The process according to claim 15, wherein
the amino acid, the amino acid derivative or the salt has a secondary amino group, where the amino acid, the amino acid derivative or salts thereof are selected from nonpolar amino acids selected from alanine, valine, methionine, leucine, isoleucine, proline, tryptophan, phenylalanine, basic amino acids comprising arginine, histidine, polar and neutral amino acids comprising tyrosine, threonine, glutamine, glycine, serine, cysteine, asparagine and/or acid amino acids selected from glutamic acid and aspartic acid, where the derivatives of the amino acids comprise the mono-, dicarboxylic acid esters, amides of the primary amino groups of the amino acids, amides of the carboxylic acid groups of the amino acids and/or esters with the primary hydroxyl groups or thioesters of the HS group of the amino acids.

21. The process according to claim 15, wherein
a diisocyanate is reacted with an amino acid derivative or salt thereof to give an amino acid isocyanate.

22. A composition obtainable by the process according to claim 15.

23. A compositions comprising the siloxane polymers according to claim 12, and mixtures thereof comprising
a) siloxane polymers of the general formula XI, and mixtures thereof or
b) siloxane polymers with a central polysiloxane polymer block B selected from
(i) at least one siloxane polymer of the general formula I, and mixtures comprising this polymer,
(ii) at least one siloxane polymer of the general formula Ia, and mixtures comprising this polymer or
(iii) at least one siloxane polymer of the general formula Ib, and mixtures comprising this polymer.

24. An intermediate for preparing siloxane polymers of the formula I according to claim 1, selected from amino acid isocyanates or salts or mixtures thereof, of formulae IXa, IXb, IXc and IXd
Q2*—NH(CO)NH-″2Q-NCO (IXa)
and/or
Q1*—NH(CO)NH-″1Q-NCO (IXb)
Q2*—NH(CO)NH-Q2″-NCO (IXc)
and/or
Q1*—NH(CO)NH-Q1″-NCO (IXd)
in particular with amino acid isocyanates of the formulae IXa*, IXb*, IXc*, IXd* or salts thereof

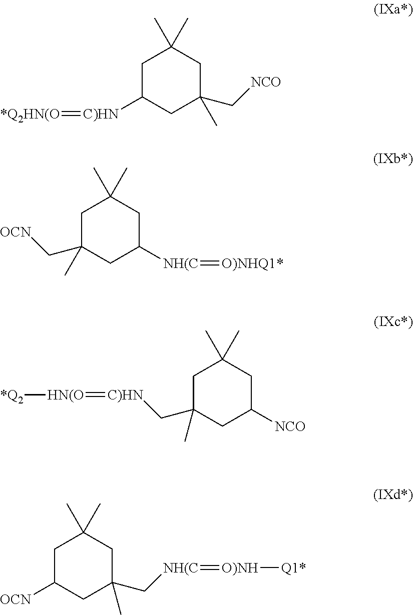

25. A formulation comprising at least one siloxane polymer according to claim 1.

26. A method of utilizing the siloxane polymers according to claim 1 as additives in cosmetic formulations, as additives in pharmaceutical formulations, in coatings, pastes, as foam stabilizers or foam additives for polyurethane foams, as hand improvers or impregnation compositions during the during the production of fibres, textiles, in cosmetic formulations for the treatment, post-treatment and protection of keratin fibres, and also skin and skin appendages, as additives in detergents, fabric softener formulations, in cosmetic formulations including creams, rinses, hair washing compositions, washing compositions, setting compositions, care rinses, care pastes, sprays, hair sprays, for improving the combability of keratin or textile fibres of natural or synthetic origin.
